(19) United States
(12) Patent Application Publication
(10) Pub. No.: US 2025/0288349 A1
(43) Pub. Date: Sep. 18, 2025

(54) PERMEABILIZATION AND ELECTROLYSIS FOR ABLATION WITH EXTRACELLULAR MATRIX RETENTION

(71) Applicant: Intuitive Surgical Operations, Inc., Sunnyvale, CA (US)

(72) Inventors: Boris RUBINSKY, El Cerrito, CA (US); Paul MIKUS, Miami, FL (US); Kyle MILLER, San Jose, CA (US); Liel RUBINSKY, El Cerrito, CA (US)

(73) Assignee: Intuitive Surgical Operations, Inc., Sunnyvale, CA (US)

(21) Appl. No.: 18/858,738

(22) PCT Filed: Apr. 21, 2023
(86) PCT No.: PCT/US2023/019480

§ 371 Date: Oct. 21, 2024

Related U.S. Application Data
(60) Provisional application No. 63/333,948, filed on Apr. 22, 2022.

(51) Int. Cl. A61B 18/14; A61B 18/00; A61B 18/12

(52) U.S. Cl. CPC A61B 18/1492; A61B 18/1206; A61B 2018/0022; A61B 2018/00494; A61B 2018/00577

(57) ABSTRACT

Apparatuses, systems, and methods are disclosed for providing controlled delivery of electrolysis products and cellular permeabilization treatment to a site in tissue. A minimally invasive regenerative surgery of subjecting a target area in living tissue to an electric input composed of a combination of electric fields of a magnitude that permeabilizes the cell membrane and to an electrolytic reaction that generates products of electrolysis of a magnitude that, by themselves, do not cause damage to cells or the extracellular matrix, but induces cell death in combination with electric field of the magnitude that permeabilizes the cell membrane. It is shown that the apparatuses, systems, and methods generate a region of tissue in which complete regeneration of the ablated tissue occurs without massive inflammation, ulceration, coagulative necrosis, fibrotic tissue, or scar tissue.

### CROSS-REFERENCE TO RELATED APPLICATION(S)

This application claims priority to U.S. Provisional Application No. 63/333,948 filed Apr. 22, 2022, which is incorporated herein by reference, in its entirety, for any purpose.

### TECHNICAL FIELD

The present disclosure relates generally to the field of tissue regeneration. Examples are described that utilize cell permeabilization and electrolysis to ablate cells while retaining the decellularized extracellular matrix to allow for tissue regeneration and tissue engineering.

### BACKGROUND

Tissues in the body may be treated in surgical or medical procedures using resection and removal. In minimally invasive tissue ablation, targeted tissues may be treated inside the body (e.g., in situ) using procedures that do not involve resection or require minimal resection. Some examples of minimally invasive tissue ablation techniques include electrolytic ablation, cryosurgery, chemical ablation (e.g., alcohol injection), and thermal ablation (e.g., radiofrequency, microwave).

Tissue engineering and regenerative medicine are relatively new areas of research in medicine and surgery. Regenerative surgery generally involves ablation of targeted cells in the treated region and then allowing or promoting the growth of desirable cells in the treated region taking advantage of the extracellular scaffold that remains intact by the programmed removal of the treated cells by the immune system.

In minimally invasive tissue ablation using cryosurgery or thermal ablation, the immune system removes the ablated tissue. However, because prior art ablation techniques affect all the molecules of the treated tissue including both the cellular contents and the extracellular matrix indiscriminately, there is no regrowth of normal tissue such as would be needed for tissue engineering and/or regenerative medicine, and instead scar tissue replaces the ablated tissue within a period of six to eight weeks. For example, after cryosurgery and thermal ablation there is initially massive inflammation where coagulative necrosis occurs. This injury eventually leads to the development of scar, ulceration, necrotic, and fibrotic tissues remaining at several weeks post-procedure with scar tissue and fibrosis being potentially permanent in these patients. Prior art minimally invasive tissue ablation processes using elevated temperatures for ablation (e.g., via RF energy) and freezing also produce scar, ulceration, necrotic, and fibrotic tissue.

### SUMMARY

Examples of methods are described herein. An example method may include positioning at least one electrode proximate tissue of a patient, applying an electric field to at least a portion of the tissue of the patient using the at least one electrode, the electric field configured to permeabilize cell membranes in a targeted tissue of the patient, thereby generating permeabilized cells, wherein the permeabilized cells are within an area targeted for ablation, and performing electrolysis to generate products of electrolysis in the tissue to ablate the permeabilized cells while leaving intact an extracellular matrix in the area targeted for ablation.

In some example methods, positioning comprises delivering a catheter to a patient, the catheter including the at least one electrode.

In some example methods, applying the electric field comprises applying a voltage between the at least one electrode and a return electrode placed proximate the patient.

In some example methods, the catheter includes a pair of electrodes including the at least one electrode, and applying the electric field comprises applying a voltage between the pair of electrodes.

In some example methods, the extracellular matrix provides a scaffold for tissue regeneration.

In some example methods, the products of electrolysis do not form scar tissue, fibrotic tissue, coagulative necrosis, ulceration, or combinations thereof.

In some example methods, applying the electric field comprises delivering pulses between 100 V and 1000 V at a capacitance of between 50 μF and 100 μF.

In some example methods, a number of pulses is between one and 10.

In some example methods, a number of pulses is selected based on a depth of ablation.

In some example methods, an electric field generated is between 450 V/cm and 850 V/cm.

In some example methods, the tissue is a mucosal layer of the small intestine.

In some example methods, the electric field at an interface between the mucosal and submucosal layer has a field strength less than that used for irreversible electroporation.

In some example methods, the electric field at the interface between the mucosal and submucosal layer is less than 850 V/cm.

In some example methods, the electric field at the interface between the mucosal and submucosal layer is between 450 V/cm and 850 V/cm.

Some example methods include introducing additional cells to the area targeted for ablation, through transplantation, migration, or combinations thereof, wherein the additional cells facilitate regeneration.

Examples of systems are described herein. An example system includes a delivery system with having a plurality of electrodes configured to contact an area of tissue targeted for ablation and a controller configured to control a charge applied to the electrodes, the controller configured to induce a voltage to generate permeabilized cells in an area of tissue targeted for ablation, and induce a current to generate products of electrolysis to cause ablation of the permeabilized cells while leaving an extracellular matrix of at least some of the permeabilized cells intact to allow for regeneration of the tissue including the permeabilized cells.

In some example systems, the delivery system comprises a catheter and a distal portion of the catheter comprises an expandable member configured to contact the area of tissue, the expandable member including the plurality of electrodes.

In some example systems, the plurality of electrodes extend from a proximal portion to a distal portion of an extendable member.

In some example systems, the plurality of electrodes is between 12 electrodes and 16 electrodes.

In some example systems, the plurality of electrodes are round and produce an electric field between 350 V/cm and 900 V/cm.

In some example systems, the electrodes have a flat surface and produce an electric field between 580 V/cm and 930 V/cm.

Some example systems may further include a power supply that delivers pulses between 100 V and 1000 V at a capacitance of between 50 μF and 100 μF.

In some example systems, the controller is configured to select a number of pulses based on a depth of ablation.

Examples of non-transitory computer-readable storage media are described herein, which may be encoded with instructions that when executed by a controller, cause the controller to induce a voltage to generate permeabilized cells in an area of tissue targeted for ablation, and induce a current to generate products of electrolysis to cause ablation of the permeabilized cells while leaving an extracellular matrix of at least some of the permeabilized cells intact to allow for regeneration of the tissue including the permeabilized cells.

In some examples, the induced voltage produces an electric field between 350 V/cm and 930 V/cm.

In some examples, a controller delivers between one and 10 pulses between 100 V and 1000 V, at a capacitance of between 50 μF and 100 μF.

In some examples, the instructions further cause the controller to select the number of pulses based on a depth of ablation.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples in accordance with the disclosure and are therefore not to be considered limiting in scope, the disclosure will be described with additional specificity and detail through use of the accompanying drawings, in which:

FIG. 1 is a schematic illustration of an electroporation system according to examples described herein.

FIG. 2A illustrates a catheter having an array of parallel electrodes arranged in a sequential polarity pairs, (+, −, +, − . . . ), the catheter depicted in an unexpanded configuration according to examples described herein.

FIG. 2B illustrates the catheter of FIG. 2A in an expanded configuration.

FIG. 3 illustrates a catheter according to one example described herein.

FIG. 4 is a schematic illustration of a system arranged according to one example described herein.

FIG. 5A is a schematic illustration of anatomy arranged in accordance with examples described herein.

FIG. 5B is a schematic illustration of anatomy arranged in accordance with examples described herein.

FIG. 6 is a cross-section schematic illustration of a small intestine with the catheter of FIG. 2B deployed in an expanded configuration in a cavity of the small intestine, in accordance with examples described herein.

FIG. 7A is a schematic of a medical procedure for inserting catheters or systems according to examples described herein.

FIG. 7B is a schematic of a medical procedure for inserting catheters or other systems according to examples described herein.

FIG. 8 is a cross-section of an ablation site of a small intestine of a pig after ablation using examples described herein.

FIG. 9 is a cross-section of an ablation site of a small intestine of a pig after ablation using examples described herein.

FIG. 10 is a cross-section of an ablation site of a small intestine of a pig after ablation using examples described herein.

FIG. 11 is a cross-section of an ablation site of a small intestine of a pig after ablation using examples described herein.

FIG. 12 is a cross-section of an ablation site of a small intestine of a pig after ablation using examples described herein.

FIG. 13 is a cross-section of an ablation site of a small intestine of a pig using examples described herein.

FIG. 14A is a cross-section of an ablation site of a small intestine of a pig after ablation using examples described herein.

FIG. 14B is a magnification of FIG. 14A.

FIG. 15A is a cross-section of a control sample of an intestine of a pig.

FIG. 15B is a cross-section of an ablation site of a small intestine of a pig after ablation using examples described herein.

FIG. 16A is a cross-section of a control sample of an intestine of a pig.

FIG. 16B is a cross-section of an ablation site of a small intestine of a pig after ablation using examples described herein.

FIG. 17A is a cross-section of an ablation site of a small intestine of a pig after ablation using examples described herein.

FIG. 17B is a cross-section of an ablation site of a small intestine of a pig after ablation using examples described herein.

FIG. 18A is a cross-section of an ablation site of a small intestine of a pig after ablation using examples described herein.

FIG. 18B is a magnification of FIG. 18A.

FIG. 18C is a magnification of FIG. 18B.

FIG. 19A is a cross-section of an ablation site of a small intestine of a pig after ablation using examples described herein.

FIG. 19B is a cross-section of an ablation site of a small intestine of a pig after ablation using examples described herein.

FIG. 19C is a cross-section of an ablation site of a small intestine of a pig after ablation using examples described herein.

FIG. 19D is a cross-section of a control sample of an intestine of a pig.

FIG. 19E is a cross-section of an ablation site of a small intestine of a pig after 24 hours after ablation using examples described herein.

FIG. 19F is a cross-section of an ablation site of a small intestine of a pig after 72 hours after ablation using examples described herein.

FIG. 20A is a cross-section of an ablation site of a small intestine of a pig after eight days after ablation using examples described herein.

FIG. 20B is a cross-section of an ablation site of a small intestine of a pig after eight days after ablation using examples described herein.

FIG. 20C is a cross-section of an ablation site of a small intestine of a pig after eight days after ablation using examples described herein.

FIG. 21 is a schematic cross-section of a duodenum illustrating an example system arranged in accordance with examples described herein during operation.

FIGS. 22A-22C are schematic illustrations of a cell undergoing electrolytic electroporation in accordance with examples described herein.

FIG. 23 is a diagram of a manipulator system according to examples described herein.

FIG. 24A illustrates an instrument system according to examples described herein.

FIG. 24B illustrates a distal portion of the instrument system of FIG. 24A with an extended example of an instrument according to examples described herein.

FIG. 25 is a perspective view of a manipulator system according to examples described herein.

FIG. 26 is a schematic view of a manipulator system according to examples described herein.

FIG. 27 is a schematic illustration of electric field amplitudes that may be used in examples of systems described herein.

FIG. 28A shows a formula representing an equation for an electric field produced by electrodes with a space charge density in examples of systems described herein.

FIG. 28B shows a formula representing an equation for the electric field linked to a potential field in examples of systems described herein.

FIG. 28C shows a formula representing an equation for an electric displacement field produced by electrodes in examples of systems described herein.

FIG. 28D shows a formula representing a relationship between the electric displacement field and the space charge density in examples of systems described herein.

FIG. 29 is a table listing relationships between numbers of electrodes, types of electrodes, and ranges of electric fields used in examples of systems described herein.

FIG. 30A shows a formula representing an equation for a potential around a cylindrical catheter and a ground pad on an outer surface in examples of systems described herein.

FIG. 30B shows a formula representing an equation for a field representing a relationship between a potential and a radius in examples of systems described herein.

FIG. 30C shows a formula representing an equation for computing potential at relationship between electric displacement field and the space charge density in examples of systems described herein.

FIGS. 31A-31F show formulas representing equations to determine electric fields for use in examples of systems described herein.

FIG. 32 is a schematic illustration of an electrolytic electroporation electrode array used in examples of systems described herein.

FIG. 33 is a table showing relationships between treatment sites, experimental conditions, and results in examples of systems described herein.

FIGS. 34A and 34B are schematic illustrations showing relationships between numbers of pulses and average ablation depths in examples of systems described herein.

FIG. 35 is a schematic illustration showing relationships between numbers of pulses and circumferential ablation percentages in examples of systems described herein.

### DETAILED DESCRIPTION

Certain details are set forth below to provide a sufficient understanding of embodiments of the disclosure. However, it will be clear to one skilled in the art that embodiments of the disclosure may be practiced without these particular details. Moreover, the particular embodiments of the present disclosure described herein are provided by way of example and should not be used to limit the scope of the invention to these particular embodiments. In other instances, well-known materials, components, processes, controller components, software, circuitry, timing diagrams, and/or anatomy have not been described or shown in detail in order to avoid unnecessarily obscuring the embodiments.

This disclosure describes the combined effect of electroporation with electrolysis that may allow for more effective ablation of tissue and regeneration of tissue after ablation. This disclosure is directed to apparatuses and systems for providing controlled delivery of cellular permeabilization treatment and electrolytic products to a targeted site in tissue. A method for minimally invasive regenerative surgery is disclosed that includes subjecting a target area in living tissue to a combination of one or more electric fields and electrolysis. The electric fields may be generated by applying voltages and/or currents between one or more pairs of electrodes. The electric fields may be generated to have a magnitude to permeabilize cell membranes in a region where ablation is desired. The electric fields may be generated to produce products of electrolysis of a magnitude that, by themselves, do not cause damage to cells or to the extracellular matrix. However, when sufficient products of electrolysis are generated in the region of permeabilized cells at parameters described herein, cellular death occurs within that region of the applied field, without damaging the extracellular matrix or scaffolding.

Accordingly, electroporation may be performed that targets the cell membrane. Electroporation permeabilizes the cell membrane. Reversible electroporation may be used in which the permeabilization may cease after the electric fields are removed. Cells may survive reversible electroporation with the pores within the membrane resealing and returning to homeostasis. In examples described herein, however, products of electrolysis may be applied to permeabilized cells, and cause cell death of the permeabilized cells within the applied electrical field. However, the magnitude of the generated products of electrolysis may leave the extracellular matrix of the permeabilized cells intact being inefficient to cause harm or ablation within the extracellular matrix. The extracellular matrix generally refers to a three-dimensional network of proteins and/or other molecules (e.g., collagen fibers, proteoglycans, and/or proteins such as fibronectin and/or laminin) that provide structure for cells and tissues. The extracellular matrix may additionally provide signaling for cell growth and development. The extracellular matrix may be referred to as a non-cellular component, but may generally surround cells in tissues. Extracellular matrices may be used as scaffolds for tissue regeneration and/or engineering. For example, cells may be regenerated, grown, transplanted, or otherwise nurtured on extracellular matrix. By retaining the extracellular matrix in regions of otherwise ablated cells described herein, cell regeneration and/or tissue engineering may occur in the region of ablated cells. Regeneration and/or tissue engineering may be facilitated in some examples by the retention of the extracellular matrix of the ablated cells. In other examples, extracellular matrix may be transplanted from a region of ablated cells to another region (which may be another region of ablated cells in some examples), allowing for regeneration and/or tissue engineering in the transplanted region. The extracellular matrix may be used for regeneration in the region where it was transplanted. Alternatively or additionally, once the extracellular matrix is present, material may be injected into the extracellular matrix to enhance regrowth (e.g., by injecting pancreatic islets in some examples). Accordingly, material may be injected into the extracellular matrix in a region of tissue ablated using electroporation and electrolysis as described herein and/or in a region to which the extracellular matrix has been transplanted.

One aspect of the ablation technology in this disclosure accordingly relates to the use of electrolysis in the process of ablation. The process of electrolysis refers to an electrochemical reaction that occurs at the electrode surfaces of electrodes in contact with an ionic conducting media (e.g., an aqueous solution). The electrochemical reaction occurs generally as a result of an electric potential driven transfer between electrons from the electrode and ions or atoms in the solution. The chemical species generated on the electrodes diffuse in the ionic conductive media from the electrodes outward in an electroosmotic diffusion process. In an environment typical of biological matter, these chemical reactions also yield changes in pH, resulting in an acidic region near the anode and a basic region near the cathode as well as the production of chemical species that can be toxic to biological matter. These products and changes in pH diffuse from the electrodes into the biological media. Electrolysis alone (e.g., without cell membrane permeabilization) is a well-known chemical ablation mechanism, and the extent of ablation is a function of the nature of the chemical species and its concentration. Because the products of electrolysis are generated at the electrodes, to ablate large volumes of biological matter, the products of electrolysis must diffuse throughout the targeted volume. Diffusion is a slow process and therefore one of the major drawbacks of electrolytic ablation using ablation from electrolysis alone is that the procedure may take a long time. The products of electrolysis and the tissue ablation treatment using electrolysis alone may occur over a period on the order of magnitude of tens of minutes to hours. This is because the toxic level concentration of products of electrolysis throughout the entire target volume to ablate that volume and because mass diffusion is a slow process. Additionally, when electrolysis alone is used to ablate tissue, the concentration and exposure time to electrolysis products are such that scarring, ulceration, coagulative necrosis, and/or fibrosis results.

Electrolysis preferentially utilizes inert electrodes that do not participate in the process of electrolysis except as a source or sink of electrons or as catalysts. When participating non-inert electrodes are used in the process they can generate ion metals that may cause systemic damage to the body, such as excess of iron or even metallic fragments. Electrolysis products may include products toxic to cells. For example, an abundance of protons H⁺ can generate a non-physiological acidic environment diffusing from the anode and an abundance of OH can generate a non-physiological basic environment diffusing from the cathode. Permeabilized cells exposed to non-physiological pH can die because the intracellular homeostasis is disrupted and the normal cell chemical pathways are disrupted. The products of electrolysis can combine and generate chemicals that are toxic to cells such as hypochlorous acid or hydrochloric acid whose effect may depend in part on the pH in the region where they form. For example, passing a current through a saline solution (NaCl and H₂O) at a pH between 3 and 5 may generate extremely toxic hypochlorous acid (HOCl). Products of electrolysis affect biological matter through chemical reactions in which they are involved, as a function of their concentration and time of exposure. High concentrations and longtime of exposure to products of electrolysis affect all the biological molecules in the targeted tissue, an undesirable effect for tissue regeneration applications. However, judiciously delivering products of electrolysis at lower concentrations for shorter periods of time at a level at which they are unable to affect all the biological molecules in a treated volume of tissue and are able to only affect cells whose membrane is temporarily or permanently permeabilized by diffusing into the cell and affecting the intracellular homeostasis can cause cell death without affecting the extracellular matrix. For example, typical methods for tissue ablation by electrolysis alone may utilize the delivery of charge on the order of 30-100 Coulombs per cm diameter of targeted tissue and typically 40 mA delivered for 90 minutes. It will be seen in examples herein that for regenerative electrolytic electroporation ablation, exponentially decaying currents may be used having decaying amplitudes for much shorter times—a peak of 250 mA delivered for less than 2 ms in one example. Accordingly, current provided for electrolysis for electrolytic electroporation ablation described herein may be provided using an exponential decay or other decaying waveform, and may have an amplitude on the order of tens or hundreds of mA. The current may be provided for a time scale on the order of milliseconds in some examples. Multiple pulses (e.g., multiple waveforms) may be used in some examples.

Examples described herein utilize electrolysis products to cause cell death together with electroporation to permeabilize the cell membrane. The electrolysis products used in these examples are sufficient to ablate (e.g., cause cell death) of permeabilized cells in a relatively short time frame. However, the concentration of electrolysis products and exposure time are insufficient to cause cell death of non-permeabilized cells and thereby do not cause or contribute to the formation of scar tissue. Rather, the electrolysis products in these examples are used to ablate permeabilized cells only, while leaving the extracellular scaffold (e.g., extracellular matrix) intact.

Examples of systems disclosed herein may include electrodes, a power supply, and a controller. The controller may control a charge delivered to the electrodes to induce one or more electric fields. For example, the electrodes may be used to generate a current to produce electrolysis products and a voltage difference to produce an electric field that induces electroporation. The duration and magnitude of the charge applied may determine the dose of the electrolytic products and the degree of the permeabilization of cells in the treatment site. Accordingly, a region of cell ablation may be determined by a region in which cells are exposed to the combination of permeabilization and to electrolysis products that cause ablation. The ablation, however, may leave the extracellular matrix intact in the region of ablated cells where the electrical field has been applied. The composition of the electrodes may be chosen in accordance with the desired products produced and electroporation effects.

Examples of systems and apparatuses described herein may include electrodes used to apply electrolysis and electroporation to tissue. Example apparatuses may be used for treating internal tissue. Examples of systems and methods described herein create a region of ablated tissue in which the extracellular matrix is retained to facilitate regeneration (including complete regeneration in some examples). The method and devices can accordingly be used for in situ regeneration of new tissue that may replace an undesirable cell type with a desirable cell type or an undesirable extracellular matrix with a desirable extracellular matrix. Examples of systems and methods can be used in combination with the implantation of a cell type in the treated tissue in some examples. Example methods and devices can also be used to generate an extracellular matrix wholly or partially devoid of living cells that can also be removed from the site of treatment and the remaining non-cellular matrix material may be implanted or transplanted into a different repair site.

Examples of systems and methods described herein utilize the combined effect of electroporation with electrolysis that may allow for more effective ablation of tissue and regeneration of tissue. Electroporation—the permeabilization of the cell membrane through the application of electric fields across cell membranes—may be used for tissue ablation with two different sets of electric parameters: those used for reversible electroporation and those used for irreversible electroporation. Reversible electroporation is the permeabilization of the cell membrane that generally ceases after the electric fields are removed and in which the cells survive the application of the electric field. In irreversible electroporation, the permeabilization of the cell membrane is permanent, leading to cell death. Accordingly, some electric parameters yield irreversible electroporation in which pores formed in the cell membrane do not reseal after the field is removed, and the permeabilization becomes permanent, resulting in electroporated cell death. In some examples, for biological tissues, electric fields lower than about 1500 V/cm to about 200 V/cm are considered to produce reversible electroporation, and electric fields higher than about 1500 V/cm are considered irreversible electroporation electric fields. In some examples, electric fields lower than about 1000 V/cm to about 350 V/cm are considered to produce reversible electroporation, and electric fields higher than about 1000 V/cm are considered irreversible electroporation electric fields. Examples of systems and methods described herein may generally utilize reversible electroporation, which may avoid disadvantages of irreversible electroporation in some examples, such disadvantages may include heating, complexity of providing such large electric fields, and muscle contractions that may result from the fields.

Electroporation generally targets the cell membrane. Because the procedure primarily affects the cell membrane, features of the extracellular matrix are spared. Since the electric fields that result in irreversible electroporation are very large, e.g., 3000 V/cm, however, the high voltages applied to produce irreversible electroporation electric fields can cause partial thermal damage damaging both cells within the field and extracellular matrix. The high voltages may also cause electric discharge across gaseous layers that form by electrolysis near the electrodes resulting in severe muscle contractions or arcing that has potential to inadvertently affect tissue in that region. The large electric fields utilized in irreversible electroporation may also cause muscle contraction necessitating muscle paralytics and general anesthesia thus prolonging procedure case times and risk to patients. Reversible electroporation generally utilizes smaller electric fields, usually less than 1500 V/cm. Reversible electroporation parameters are generally utilized in examples described herein.

Electrolysis may generate electrolysis products that may be used to ablate permeabilized cells. However, electrolysis products applied to non-permeabilized cells in sufficient quantity for sufficient time to ablate non-permeabilized cells may generate scar, ulceration, or fibrotic tissue. Scar tissue and fibrosis is an indication that the extracellular matrix has been affected, and the ability for cells to regenerate and/or tissue engineering to occur in the ablated region may be inhibited. Accordingly, examples described herein provide exposure to electrolysis products at sufficient levels and time to cause cell death of permeabilized cells, but low enough to leave the extracellular matrix intact in the region of the permeabilized cells. In addition, the level of electrolysis products and/or exposure time applied are insufficient to ablate non-permeabilized cells and/or to cause the generation of scar, ulceration, or fibrotic tissue.

For example, tissue engineering generally refers to combining cells, scaffolds, and/or growth factors to regenerate tissues or replace damaged or diseased tissues. Regenerative medicine generally combines tissue engineering with other strategies, including cell-based therapy, gene therapy, and immunomodulation, to induce in vivo tissue/organ regeneration. In regenerative medicine it is important that the treated tissue remains capable of regeneration. The formation of scar tissue may inhibit and/or prevent tissue regeneration.

Examples of systems and methods described herein utilize reversible electroporation to permeabilize cells within a targeted area for ablation. The fields used to generate reversible electroporation and/or other electric fields may be used to produce products of electrolysis. The products of electrolysis are introduced into the targeted area to cause ablation of the permeabilized cells, only (e.g., through diffusion across the permeabilized membrane), without affecting non-permeabilized cells and the extracellular matrix. The quantity, concentration, and strength of the electrolysis products provided are such that the extracellular matrix in the region of the permeabilized, ablated cells remains intact and that non-permeabilized cells survive the treatment. The formation of major inflammation, ulceration, and necrosis is avoided, and ultimately scar and/or fibrosis tissue is drastically reduced and/or eliminated. Accordingly, tissue may be regenerated on the decellularized extracellular matrix generated by the combination of cell membrane permeabilization and application of products of electrolysis.

It is to be understood that, although systems and methods described herein utilize reversible electroporation, there may occur localities or incidental areas where, while the majority of the tissue is affected by reversible electroporation, conditions are such that irreversible electroporation, or even thermal ablation, may occur in limited parts of the treated tissue. Generally, examples of systems and methods described herein primarily utilize reversible electroporation—e.g., more, or the majority of, ablation occurs through the use of reversible electroporation and exposure to electrolysis products rather than by irreversible electroporation and/or thermal ablation. Further, in some embodiments, substantially all of the affected tissue is affected by reversible electroporation, and in other embodiments, all of the affected tissue is affected by reversible electroporation.

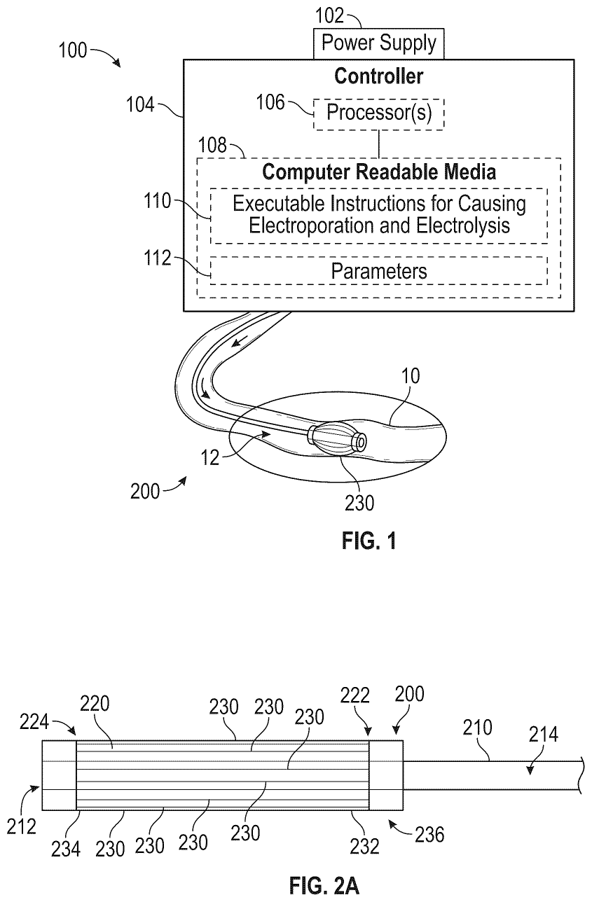

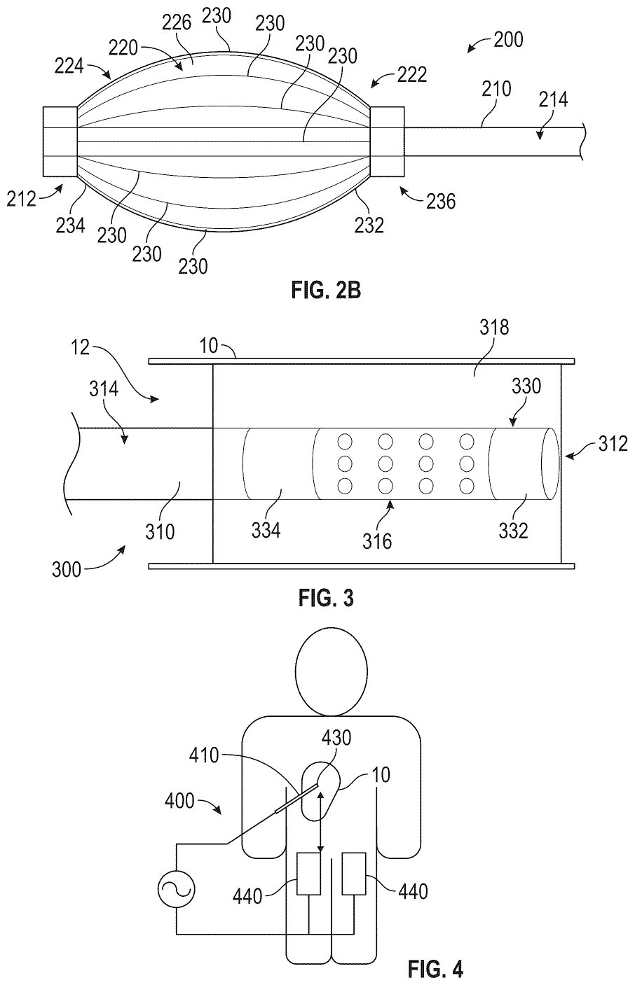

FIG. 1 is a schematic illustration of an electrolytic electroporation electrode system 100 arranged in accordance with examples described herein. Generally, examples of systems described herein may include a delivery system and a controller. In the example of FIG. 1, the system 100 includes a controller 104 and an electrolytic electroporation electrode catheter 200. Examples of catheters that may be used to implement the catheter 200 are described in further detail in regard to FIGS. 2A and 2B. The controller 104 may include a processor 106, computer-readable media 108 (e.g., memory), and other computing system components, such as one or more input devices, output devices, sensors, and/or communication devices in some examples. Additional, fewer, and/or different components may be used in other examples. The computer-readable media 108 includes executable instructions for causing electroporation and electrolysis 110 with the catheter 200. The computer-readable media 108 may include stored parameters 112 that may be used in the process for causing electroporation and electrolysis, such as electric field strengths, voltage and/or current levels, capacitance, waveform shapes, a number of pulses, and/or exposure duration parameters.

The controller 104 may be implemented using a computing device. Examples of computing devices include controllers, microcontrollers, computers, servers, medical devices, smart phones, tablets, wearable devices, and the like. The computing device may be handheld and may have other uses as well.

The controller 104 may include one or more processors, such as the processor 106. Any kind or number of processors may be present, including one or more central processing unit(s) (CPUs) and/or graphics processing unit(s) (GPUs) having any number of cores, controllers, microcontrollers, and/or custom circuitry such as one or more application specific integrated circuits (ASICs) and/or field programmable gate arrays (FPGAs).

The controller 104 described herein may include the computer-readable media 108, such as memory. Any type or kind of memory may be present (e.g., read only memory (ROM), random access memory (RAM), solid state drive (SSD), secure digital card (SD card), and the like). While a single box is depicted as the computer-readable media 108 in FIG. 1, any number of computer-readable media 108 devices may be present. The computer-readable media 108 may be in communication with (e.g., electrically connected to) the processor 106.

The computer-readable media 108 may store executable instructions for execution by the processor 106, such as executable instructions for causing electroporation and electrolysis 110 with the catheter 200 utilizing stored parameters 112 for the catheter 200. In this manner, techniques for applying electroporation and electrolysis in tissue may be implemented herein wholly or partially in software.

The executable instructions may include instructions to control a charge delivered to electrodes, such as electrodes 230 of the catheter 200. Accordingly, the controller 104 may induce a voltage difference across the targeted tissue to generate an electric field that causes permeabilization of cells in an area of tissue targeted for treatment. In the illustrated embodiment, a plurality of electrodes 230 are disposed on a distal end portion of the catheter 200. The plurality of electrodes 230 are illustrated in a cavity or lumen 12 formed by a tissue 10. Although the catheter 200 is shown disposed within a cavity 12 of a tissue 10, the catheter 200 may be on the surface of the tissue 10, inside the tissue 10, and/or proximate to the tissue 10. Moreover, although a catheter is shown being used to position the electrodes used for permeabilization and/or the generation of electrolysis products, in other embodiments, other delivery systems may be used and/or the electrode(s) may be positioned proximate the tissue in other ways—e.g., by contacting the tissue with electrode(s) or using a probe, a pad, needle electrodes, flexible laparoscopic electrodes, or another device coupled to the electrodes to bring the electrodes proximate the tissue.

Generally, electrodes used in examples described herein may be used in a monopolar configuration, or a bipolar configuration, or combinations thereof. Generally, a monopolar configuration may include an active electrode (e.g., an electrode on or in the surgical field) and a return electrode. The return electrode may be placed outside the surgical field but in contact with the patient in some examples (e.g., using a pad having an electrode). In this manner, one polarity (e.g., the polarity of the active electrode) is in the surgical field. Accordingly, a monopolar configuration of electrodes may include a pair of electrodes—with one energized and one serving as a return. The return electrode may not be provided on a same device as the energized electrode. For example, the return electrode may be provided on a pad placed proximate the tissue. In a bipolar configuration, current may travel from one electrode of a pair to another electrode of a pair. The electrodes in the pair may accordingly be said to be of opposite polarity. Multiple pairs of electrodes may be used. In a monopolar configuration, multiple active electrodes may be present and may pass current through a shared return electrode in some examples, or through respective return electrodes in other examples. In a bipolar configuration, current may be passed through multiple pairs of electrodes. Any number of electrodes may be used, including two, three, four, five, six, seven, eight, nine, 10, 11, 12, 13, 14, 15, 16, 24, or 32 electrodes. Any electrode configuration may be used. In some examples, a centrally positioned electrode may be used as an anode, and multiple electrodes may be positioned about the anode to serve as a cathode. A controller, such as the controller 104 of FIG. 1, may active one or more selected electrodes in order to provide an electric field as described herein. In some examples, the controller may alternate or otherwise select a pattern of activated electrodes (e.g., activating pairs of electrodes in sequence) to shape or deliver a particular electric field. In some embodiments, one or more of the electrodes used to apply electroporation may also be used to generate products of electrolysis (e.g., some or all of the electrodes may be used for both electroporation and electrolysis), while in other embodiments, the electrodes used to apply electroporation may be different than the electrodes used to generate products of electrolysis.

Generally any shape electrodes may be used including circular, square, rectangular, or other shapes. Interdigitated electrodes may be used.

The controller 104 may also be used to induce a current through the tissue, such as between electrodes, to generate products of electrolysis. The products of electrolysis may cause ablation of the permeabilized cells. The products of electrolysis may be insufficient to destroy the extracellular matrix in the region of the permeabilized cells, and accordingly the permeabilized cells may be ablated while leaving an extracellular matrix in the region intact. The intact extracellular matrix may allow for regeneration of the tissue and tissue engineering.

The system 100 may include a power supply 102. The power supply 102 may be coupled to the controller 104. The power supply 102 may be implemented using one or more AC power sources, DC power sources, batteries, and/or waveform generators. The power supply 102 may supply power to one or more electrodes to generate a voltage and/or current and, therefore, an electric field and/or electrolysis products in the tissue. In some examples, the power supply 102 may be implemented using a signal generator, such as an exponential decay wave generator (by way of example, a Harvard Apparatus BTX 630); however, this disclosure is not limited thereto or thereby. In some examples, a signal generator may include a bank of capacitors to select from, as controlled by the controller 104. The signal generator may allow for selection of a specific charge (e.g., capacitance) per application of the charge.

The controller 104 may control the timing, strength, and duration of electric fields and/or electrolysis products provided by the catheter 200. The controller 104 may, for example, be programmed to provide an electronic signal to the catheter 200. The electronic signal may be indicative of a dose of treatment, for example, a dose of electrolysis products. The electronic signal may control the timing and magnitude of a current generated by the catheter 200 to generate an electric field. This may allow a user to customize treatment of the tissue 10. In some embodiments, the controller is coupled to a power supply 102. In some embodiments, the power supply 102 may be included in the system 100. In some embodiments, the power supply 102 is integrated with the controller 104.

Although shown as a separate component coupled to the catheter 200, in some embodiments, the controller 104 may be integrated into the catheter 200. In some embodiments, the controller 104 may include programmable circuitry coupled to the catheter 200. The controller 104 may be coupled by a wire or communicate with the catheter 200 wirelessly.

In some embodiments, the controller 104 may be programmed to provide an electronic signal indicative of a dose of the electrolysis products and/or permeability level of cell. The controller 104 may, for example, include such a program, or include one or more processing devices (e.g., processors) coupled to the computer-readable media 108 encoded with executable instructions for causing electroporation and electrolysis 110.

Examples of voltages, currents, time durations and/or time constants, electric field strengths, capacitance, and/or a number of pulses may be calculated and/or determined in accordance with methods described herein. In some examples, parameters, such as parameters 112 of FIG. 1, may be determined based on measurements taken in the tissue of interest, or a different sample of similar tissue. For example, measurements may be taken at various voltage levels with particular electrode configurations, and a voltage level, current, pulse pattern, time constant, and other factors may be identified that cause reversible electroporation and the delivery of electrolysis products to result in cell death of the permeabilized cells. Generally, electric fields may be generated in tissue that may cause reversible electroporation of cells in a target ablation area. Electrolysis products may be generated and may diffuse for a time to cause ablation of the permeabilized cells, but to leave intact the extracellular matrix in the region of the ablated cells.

Examples of parameters that may be used include a delivery of between one and 10 or more voltage pulses between 100 V and 1000 V. Those pulses may be delivered in a system in some examples having a capacitance between 1 μF and 400 μF as selected by the signal generator. In some examples other capacitance values may be used. In some examples the pulses may be delivered using a resistance of between 15-20 ohms, for example. Other resistances may be used in other examples. In some examples, electric fields between about 1500 V/cm and about 0 V/cm may be generated in tissue. A quantity of electrolysis products generated may be related to the delivered charge in Coulombs. There are several ways to calculate the delivered charge. For example, the stored electric charge in a capacitor, Q (in Coulombs, abbreviated C) is generally equal to the product of the capacitance C (in Farads, abbreviated F) of the capacitor, and the voltage V (in volts, abbreviated V) across its terminals. That is, Q=C·V. The stored electric charge is generally equal to the product of the current I (in amperes, abbreviated A) and the time t (in seconds, abbreviated s). That is, Q=I·t. By defining the capacitance and the voltage across the capacitance, the charge may be defined, and accordingly the electrolysis performance determined. When a capacitor is discharged, the capacitor generates current and the current multiplied by time must be equal to the total charge in the capacitor. When a capacitor is being discharged the current is not constant—the current decays exponentially. Therefore the time measure is given as the exponential decay time constant. The capacitance that controls the time constant is generally obtained in examples described herein from capacitors incorporated in the power supply, such as power supply 102 of FIG. 1.

In some embodiments, the time constant (e.g., exponential decay time constant of the capacitive discharge) may be between 1.7 ms and 1.8 ms. In some examples, the time constant may range from 50 us to 3 ms. In some examples, the time constant may range from 1 microsecond to 1 second, 100 milliseconds to 10 microseconds in some examples. The time constants can generally range from microseconds to milliseconds. Generally, parameters used to ablate tissue using electroporation and electrolysis techniques described herein may utilize significantly less charge per volume of tissue targeted for treatment than when electrolysis alone is used for ablation (in addition, electrolysis alone may cause scarring and/or other impediments to tissue regeneration). As an example, consider that in example experiments described herein, 500 V may be delivered in an exponential decaying waveform across a resistance of about 20 ohm with a time constant of about 2 ms. The maximal charge in some examples delivered across a resistance of 20 ohm is 25 A×(0.002) s=0.05 Coulombs. In some experiments, ablation has occurred to a depth of 2 mm around a tube with an outer diameter of 3 cm and length of 3 cm, e.g., a full ablation depth volume of 2.7 cm³. Accordingly, example electrolytic applications may utilize a ratio of the charge to the treated volume, approximately 0.18 Coulombs per cm³. Other ratios or values may be used in other examples. In contrast, examples of pure electrolytic ablation (without electroporation) may use 30-50 Coulombs per cm³. Therefore to ablate this volume of tissue by electrolysis alone may utilize orders of magnitude of charge (e.g. 50-200 Coulombs) over what is used in examples described herein. Accordingly, examples of methods and systems described herein may deliver less than 30 Coulombs per cm³ to perform electrolysis, less than 10 Coulombs per cm³ in some examples, less than 5 Coulombs per cm³ in some examples, less than 1 Coulombs per cm³ in some examples, and less than 0.5 Coulombs per cm³ in some examples. The lower limit of the time constant is generally related to a time sufficient to ensure electrolytic species (e.g., products of electrolysis) permeate the targeted area of permeabilized cells. The upper limit of the time constant is generally related to the production of electrolytic species (e.g., products of electrolysis) that can cause ablation on their own. Accordingly, electrolysis should be provided for an amount of time sufficient to allow diffusion of electrolysis products through a region of permeabilized cells. However, the amount of time electrolysis is provided should be insufficient to generate products of electrolysis that themselves cause ablation of non-permeabilized cells, and accordingly the products of electrolysis generated using electrolytic electroporation should be insufficient to damage to the extracellular matrix in the region of the ablated cells.

An electric field may be generated that is between 100 V/cm and 3500 V/cm. In some examples, the electric field may be between 100 V/cm and 1500 V/m. The electric field may be less than 1400 V/cm in some examples, less than 1300 V/cm in some examples, less than 1000 V/cm in some examples, less than 800 V/cm in some examples, and less than 600 V/cm in some examples. In some embodiments, the electric field may be between 200 V/cm and 850 V/cm. Other parameters may be used in other examples. The parameters may be stored in the computer-readable media 108 as parameters 112. The controller 104 in some examples may be used to calculate the parameters 112. In other examples, the parameters may be calculated by another system and may be provided to and/or stored by the controller 104.

The system 100 may further include one or more sensors (not shown) for measurement of pH, electric field strength, and/or other properties of the tissue 10. For example, a pH sensor may be provided. The pH sensor may in some examples be located on and/or attached to the delivery system, such as the catheter 200 of FIG. 1. In some examples, a pH sensor may be positioned near electrodes of the delivery system, such as electrodes 230 of FIG. 1. A pH value near the electrodes may accordingly be detected. The pH sensor may be coupled to the controller 104, and the detected pH value provided to the controller. Additionally or instead, a pH sensor may be provided at an outer edge of a targeted region of tissue. The pH sensor may be coupled to the controller 104, and the detected pH value provided to the controller 104. Additionally or instead, a pH sensor may be provided at a particular site in the tissue to detect pH at the particular location, such as a location at which tissue damage is not desired. The pH sensor may be coupled to the controller 104, and the detected pH value provided to the controller 104. The controller 104 may utilize one or more received pH values as an indication of tissue ablation and/or potentially damaging pH levels that may cause, or be close to causing, tissue damage. The controller 104 may combine the pH values in any manner. For example, the controller 104 may take a difference between received pH values (e.g., a pH value near an edge of a targeted tissue region and a pH value near an electrode). The controller 104 may adjust the voltage, current, and/or electric field applied to the tissue responsive to the pH level or combination of pH levels. For example, if a pH value at a location where tissue damage is not desired is at or beyond a threshold for tissue damage, the controller 104 may reduce a magnitude of electric field or a duration between pulses, or cease application of the electric field. In some examples, if a pH value in a region where tissue ablation is desired is at or beyond a threshold for tissue ablation, the controller 104 may cease application of current through electrodes immediately and/or after a desired elapsed electrolysis time to cease the electrolysis process.

A resistivity meter may be used to determine a resistance of the target tissue. A resistivity meter may be provided, for example, on or otherwise coupled to the delivery system. For example, the controller 104 and/or power supply 102 of FIG. 1 may provide an impedance measurement. The impedance measurement may determine a resistivity of the tissue contacted by electrodes of the system. For example, the controller 104 and/or power supply 102 may provide a nominal amount of current, such as DC current, through the tissue and receive a resistivity measurement and/or calculate resistivity of the tissue. In some examples, an applied voltage, current, capacitance, and/or electric field may be selected, determined, and/or allowed based on a measured resistance of the tissue. In some examples, a number of pulses of applied voltage may be selected, determined, and/or otherwise used based on a measured resistance of the tissue. In some examples, a number of pulses may range from one to 10, 20 or 30 in some examples.

A number of pulses may be selected to provide a particular dose (e.g., surface charge) that may control a depth of ablation and/or a ratio of circumferential ablation (e.g., an amount of ablation within a particular circumference may increase). For example, a delivered charge for a number of pulses may be calculated. For example, the electric charge provided from a capacitor for the number of pulses, Q (in Coulombs, abbreviated C) may generally be equal to the product of the capacitance C (in Farads, abbreviated F) of the capacitor, the voltage V (in volts, abbreviated V) across its terminals, and a number of pulses N (is a natural number from one to 10, 20 or 30, or more in some examples). That is, Q=C·V·N. By selecting a number of pulses, a depth of ablation and/or a ratio of circumferential ablation may be controlled. Devices described herein may control a number of delivered pulses (e.g., voltage pulses) based on a particular depth and/or ratio of circumferential ablation. Generally, the amount of circumference of tissue affected by ablation may increase with an increased number of pulses applied. Despite increasing ablation, the generation of heat may be reduced and/or avoided by use of the combination of reversible electroporation and electrolysis. The surface charge applied may be a fraction of the surface charge typically used if only electrolysis were used to achieve ablation.

In some examples a sensor for detecting and/or determining electric field strength may be used. The strength of the electric field at any point is found by measuring the potential difference between adjacent equipotential lines and dividing by the distance between them. The distance between the lines is taken along the electric field lines that are perpendicular to the equipotential lines. Gauss meters and/or Tesla meters may be used for this purpose in some examples.

During operation, electrodes may be brought into proximity of tissue. The electrodes may contact the tissue, be implanted in the tissue, or be positioned on, adjacent to, or near the tissue. For example, a catheter including one or more electrodes, such as the catheter 200 including electrodes 230, may be delivered to a patient to bring the electrodes proximate tissue.

Any of a variety of tissue may be treated using systems and examples described herein. Generally, tissue may be treated where tissue regeneration is desirable or where it is desirable to replace one type of cells with another. Examples include intestine, duodenum, stomach, bladder, uterus, endometrial lining, endobronchial lining, ovaries, colon, rectum, sinuses, ducts, ureters, prostate, skin, muscle, nerve, diaphragm, momentum, kidney, follicles, brain, lymphatic vessels, blood vessels, breast, esophagus, lung, liver, kidney, lymph nodes, lymph node basins, and/or heart. Generally, any endoluminal structure may be treated using systems, devices, and techniques described herein. Replacement of one type of tissue with another may be in fibrotic areas where it is desired to replace fibrotic cells with stem cells that can remodulate the area or when pancreatic islets are injected in part of the liver to generate new sources of insulin. Other tissue may be treated in other examples.

An electric field may be applied to at least a portion of the tissue using the electrode(s). For example, the controller 104 may apply voltages to the electrodes 230 to apply the electric field. In some examples, fluids or other substances may be injected into, brought into contact with, or otherwise placed in or around the tissue to aid in shaping the electric field generated in the tissue. For example, conductive fluids may aid in shaping the field (e.g., by extending the field). For example, non-conductive fluids may aid in shaping the field (e.g., by attenuating the field). In some examples, non-conductive fluids or other substances may be injected or otherwise placed in tissue to protect areas where ablation is not desired. The electric field may not penetrate and/or not be carried through the non-conductive fluid, such that the field would not reach tissue where ablation is not desired, or at least be present in insufficient strength to cause permeabilization or other cellular change.

An electric field may be applied via a plurality of surgical end effectors to target anatomy in order to provide precise, accurate, and repeatable ablation in open, laparoscopic, thoracoscopic, and/or robotically assisted procedures. For gastrointestinal applications, the delivery system (e.g., ablation instrument) delivering electrolytic electroporation may be delivered via a manual or robotic delivery device endoluminally through a trans-oral or trans-anal approach or trans-abdominally with integrated bipolar instrumentation, drop-in probes, or via catheters. For urological applications, the ablation instrument delivering electrolytic electroporation may be delivered via a manual or robotic delivery device endoluminally through a trans-urethral, trans-perineal, pre-peritoneal, or trans-abdominal approach with integrated bipolar instrumentation, drop-in probes, or via catheters. For gynecological applications, the ablation instrument delivering electrolytic electroporation may be delivered via a manual or robotic delivery device endoluminally through a trans-vaginal, trans-perineal, or trans-abdominal approach with integrated bipolar instrumentation, drop-in probes, or via catheters. For hepatobiliary applications, the ablation instrument delivering electrolytic electroporation may be delivered via a manual or robotic delivery device endoluminally through a trans-oral approach to reach the ampulla or to go externally into the liver via a trans-gastrointestinal wall route or a trans-abdominal approach with integrated bipolar instrumentation, drop-in probes, or via catheters. For neurovascular applications, the ablation instrument delivering electrolytic electroporation may be delivered via a manual or robotic delivery device through an endovascular approach or through a key hole craniotomy with integrated bipolar instrumentation, drop-in probes, or via catheters. For cardiac applications, the ablation instrument delivering electrolytic electroporation may be delivered via a manual or robotic delivery device through an endovascular or a trans-thoracic approach with integrated bipolar instrumentation, drop-in probes, or via catheters. In case of endobronchial ablation in a lung for treatment of chronic obstructive pulmonary disease, such as chronic bronchitis and emphysema, the ablation instrument delivering electrolytic electroporation may be delivered via a manual or robotic delivery device endoluminally through a trans-nasal or trans-oral approach with integrated bipolar instrumentation, drop-in probes, or via catheters. Various manual and robotic delivery devices are described further herein.

The electric field may be of a strength in the tissue to cause reversible electroporation in a region of cells targeted for ablation. Accordingly, cell membranes in an area of tissue targeted for ablation may be permeabilized. In the example of FIG. 1, the circle depicted around an end of the catheter 200 may indicate a region of cells that may be permeabilized according to an electric field applied by the electrodes. For example, Example 1 below describes a 30 mm diameter expanded electrode configuration with a 0.85 mm deep field. The electric field may be constant for a time in some examples and/or may be pulsed. The pulses of the electric field may have any of a variety of shapes (e.g., square pulses, triangular pulses, impulse pulses, and/or exponential decay pulses).

Electrolysis may be performed to generate products of electrolysis. Electrolysis products may be generated, for example, from ions and molecules of an aqueous solution. The aqueous solution may be the native physiological concentration solution present in the tissue. The ionic composition of bodily fluids may be used as an ionic conductive media to cause the electrochemical reaction forming the basis of electrolysis and/or may be introduced to (e.g., injected into) the tissue during methods described herein. Electrolysis products may be generated by passing a current through tissue using electrodes described herein, such as electrodes 230 of FIG. 1. The electrolysis products may diffuse in the tissue and may ablate permeabilized cells. The time duration during which electrolysis is performed and/or the quantity of electrolysis products generated may be set herein such that the electrolysis products cause ablation of permeabilized cells, but not ablation of non-permeabilized cells. Moreover, the time duration and/or quantity of electrolysis products may be set such that the extracellular matrix of the permeabilized cells remains intact, which may facilitate regeneration in the region of the ablated tissue.

The process of applying the electric field to cause permeabilization and performing electrolysis may be controlled by computing systems described herein, such as the controller 104 of FIG. 1 in accordance with the executable instructions for causing electroporation and electrolysis 110.

In some examples, the electrodes may be moved to other tissue locations (e.g., by advancing and/or retracting the catheter 200). In this manner, electroporation and electrolysis may be performed at multiple locations in a patient, for example to cover a larger area. In some examples, multiple sets of electrodes may be positioned at multiple respective tissue sites such that electroporation and electrolysis may be performed at each site in parallel, reducing and/or eliminating a need to repeat the procedure as a catheter is moved through the patient.

Following ablation using electroporation and electrolysis that leaves the extracellular matrix (e.g., extracellular scaffold) intact, tissue regeneration may occur. Regeneration of tissue may occur over hours, and/or days, and/or weeks, and/or months in some examples. The tissue may itself regenerate from stem cells brought to that area by the blood circulation or from stem cells at the margins of the ablated tissue or from residual stem cells, and no additional intervention may be needed for regeneration. In some examples, however, cells or other components may be introduced to the ablated sites. For example, stem cells, organoids, or other desired cells, moieties, compounds, or components may be introduced to the ablated sites (e.g., using the catheter 200 or otherwise injecting or introducing the components). The cells or other components may facilitate (e.g., enhance) regeneration and/or may direct regeneration to a particular cell type or location.

In some examples, the migration, implantation, and/or transplantation of cells may be used to facilitate tissue engineering and/or regeneration. For example, stem cells may remain at the margin of an ablated tissue region. The stem cells in the marginal region may migrate to facilitate regeneration. In some examples, cellular signaling (e.g., in the extracellular matrix) may be promoted that may facilitate the migration or direction of cell growth.

There are several ways to regenerate desirable tissue to replace some or all of the cells ablated with electrolytic electroporation. The process may depend on the action of the immune system and the normal regeneration of cells in the targeted region. In some examples, the immune system may remove the cells within 24 hours after the application of electrolytic electroporation and at 24 hours cells begin to regenerate in the ablated area. Natural regeneration can occur in several ways. First, in some examples, cells may be ablated in a given treated area in such a way that the stem cells survive. For example, resident stem cells in the crypt of the mucosa can be spared ablation. A second way, particularly effective for areas such as the intestines, is that the resident stem cells from the margin of the ablated area proliferate along the intact extracellular matrix into the ablated area. A third way is to ensure that the blood circulation in the treated area remains intact and circulating stem cells reach the treated area through the blood circulation. This modality is particular effective in highly vascular tissues, such as the liver. Any or all of these mechanisms may be used to regenerate tissue in examples described herein. Differentiation of cells, such as hepatocytes can also aid in regeneration. An intact extracellular matrix may be central to the regeneration process. Stem cells hold great promise in treating many diseases either through promoting endogenous cell repair or through direct cell transplants. The extracellular matrix (ECM) is one component involved in mediating stem cell fate.

Desirable regeneration can be achieved through natural processes in which systemic mechanisms repopulate the decellularized extracellular matrix. However, there are other ways to repopulate the extracellular matrix with cells that are different from the original cells at that location, for example in the intestines. If the previous cells have suffered a genetic malformation, for example, genetically engineered cells may be injected into the decellularized mucosa (e.g., into a region of ablated cells) that do not carry the genetic defect to replace the original cells. For example, in the liver, in a fibrotic region, in some examples, hepatic stem cells may be injected into a decellularized fibrotic region (e.g., an ablated region) to reorganize the extracellular matrix. Other examples include pancreatic islets. A common method for treating diabetes type I is to inject extraneous pancreatic islets through the portal vein in the hope that they will implant in the liver. A preferable method is to inject the pancreatic islets in a decellularized volume of the liver. An aspect of this mode of tissue regeneration is the timeline between the ablation of cells, the removal of the ablated cells by the immune system, and the native regeneration process. In this treatment modality it would be preferential to inject the exogeneous cells in the time interval between the time when the immune system has removed the cells, so as to avoid the active immune system from attacking the implanted cells, and when the normal regeneration begins to occur, so as to avoid competition between the two types of cells. In examples described herein, this window in time may be between 6 hours after ablation and 24 hours after ablation, preferentially at about 25 hours after ablation. This window in time may be tissue specific and may be adjusted to the particular application.

FIG. 21 is a schematic cross-section of a duodenum illustrating an example system arranged in accordance with examples described herein during operation. FIG. 21 includes an expandable member 2108 disposed in a duodenum 2114. For example, in FIG. 21, the expandable member 2108 may be disposed in the fourth portion of the duodenum 2114. The fourth part of the duodenum 2114 is generally the ascending part of the duodenum, generally located at vertebral level L-3, and that may pass directly on top or slightly to the left of the aorta. However, the expandable member 2108 may alternatively be positioned in the other portions of the duodenum 2114. The expandable member 2108 may include an electrode array 2110. Expansion of the expandable member 2108 may result in the expandable member 2108 contacting an inner lining of the intestine, which may be wholly and/or partially diseased. For example, a mucosal lining 2112 may be targeted for ablation. Note that a variety of tissue structures are adjacent and/or proximal to the targeted tissue such as a pancreas 2104, a bile duct 2102, a superior mesenteric artery and vein 2106, and/or an ampulla of Vaster 2116. Examples described herein may allow for ablation and/or regeneration of the mucosal lining 2112 while minimizing and/or avoiding an effect on other structures such as vessels, ducts, and/or nerves. For example, in some embodiments, electrolytic electroporation may be applied to the mucosal lining 2112 of the duodenum 2114 without requiring sensitive structures (such as vessels, ducts, and/or nerves) to be protected from the ablation. The expandable member 2108 and/or electrode array 2110 may be used to implement and/or implemented by generally any expandable members and/or electrode structures described herein, including those shown and/or described with reference to FIGS. 1-4.

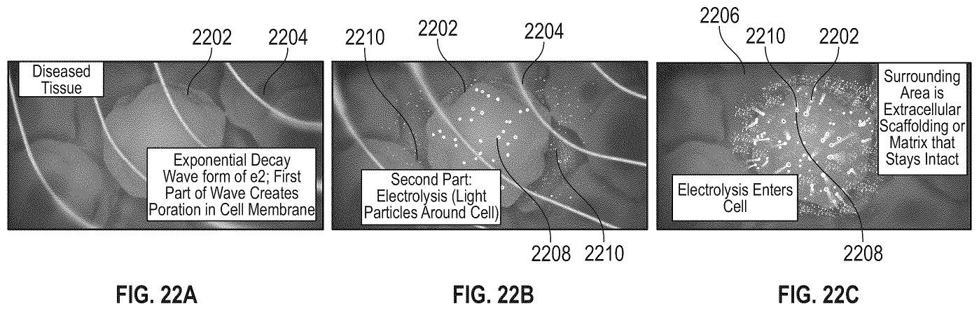

FIGS. 22A-22C are schematic illustrations of a cell undergoing electrolytic electroporation in accordance with examples described herein. In FIG. 22A, a cell 2202 is exposed to an electric field, as illustrated in part by electric field line 2204. The cell 2202 may be a portion of the mucosal lining 2112 of FIG. 21, for example. When exposed to an electric field, during an exponential decay time of the voltage and/or current waveform used to generate the field, the cell 2202 may experience electroporation. For example, holes or other perforations may be formed in the cell membrane of the cell 2202. In FIG. 22B, the cell membrane of cell 2202 is illustrated with holes 2208, indicating the membrane has become partially and/or wholly permeable. The electric field application may continue, including as shown by electric field line 2204. Electrolysis products may be generated, as illustrated by particles 2210 shown around the cell 2202 in FIG. 22B. In FIG. 22C, the cell 2202 is depicted having electrolysis products enter the cell 2202, e.g., through diffusion through the permeable cell membrane. The extracellular matrix 2206 may be unaffected by the electrolysis products, electroporation, and/or electric field application. The cell 2202 may accordingly die after exposure to the electrolysis products diffused to be within the cell membrane of the cell 2022. The dead cell may be referred to as a “ghost cell” and may be ablated. The dead cell may be absorbed by the body and/or surrounding tissue (e.g., using an immune response). In some examples, the dead cell may become detached from the submucosal layer and may be removed. Accordingly, the mucosal lining 2112 of FIG. 21 may be ablated. Following ablation, regeneration may occur, providing a healthy regenerated mucosal lining 2112.

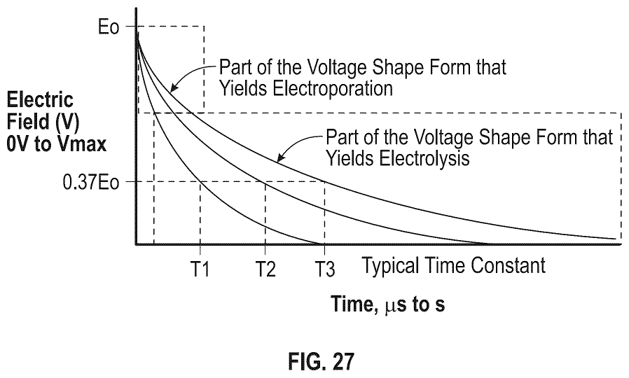

FIG. 27 is a schematic illustration of electric field amplitudes that may be used in examples of systems described herein. The electric field waveforms shown in FIG. 27 may be delivered by ablation devices described herein, such as system 100 of FIG. 1. The electric fields shown in FIG. 27 may, for example, be provided by the electrodes 230 of FIG. 1. Generally, the waveforms of FIG. 27 may represent voltages that may be applied by electrodes described herein. An exponentially decaying waveform may be used. Three examples of such a waveform with different time constants are shown in FIG. 27. The time constant refers to a time when the exponential decay has caused the level to reduce to a fraction of the peak field application. In the example of FIG. 27, the peak electric field strength is shown as E₀ and the time constant is measured at a time when the field strength has declined to 0.37E₀. The peak electric field strength (e.g., the peak voltage application) may be set, for example by the controller 104 of FIG. 1. The peak electric field strength (e.g., peak voltage to apply) may be stored in the computer-readable media of FIG. 1 in some examples. The time constant used in systems described herein may generally be on the order of microseconds to milliseconds and/or seconds. An initial portion of the waveform, generally the higher electric field strength, is typically when electroporation of cells may occur, as illustrated in FIG. 22A. A second portion of the waveform, as the strength continues to decay, is generally when electrolysis products have formed, as shown in FIG. 22B. Three waveforms are shown in FIG. 27. Each may be referred to as a pulse described herein. The three waveforms differ in their time constants (e.g., their rate of decay).

FIGS. 2A and 2B illustrate an example of the catheter 200 according to examples described herein. The catheter 200 may provide electrolysis and electroporation treatment according to the embodiments of the present disclosure. The catheter 200 includes an elongate member 210 with a distal end 212 and a proximal end (not shown). The elongate member 210 may be a tubular member that includes a lumen 214 that extends from the proximal end to the distal end 212. In some embodiments, the lumen 214 may extend from the proximal end to a position proximal to the distal end 212. The distal end 212 may have a closed end. The length of the elongate member may be at least 85 cm with a preferred length of 1.8 m and the diameter of the elongate member may be about 7 mm with a preferred maximum outer diameter of 5 mm to allow for introduction through the working channel of an endoscope.

The catheter 200 may include an expandable member 220. In the illustrated embodiment, the expandable member 220 is a balloon. Other expandable members may be used in other examples, such as meshes and the like. The expandable member 220 may be expanded in a variety of different manners. In some embodiments, the expandable member 220 may be inflated with a fluid, such as air, saline, a radiopaque solution, and the like. The fluid may be introduced to the expandable member 220 through the lumen 214 of the elongate member 210. FIG. 2A illustrates the expanded member 220 in an unexpanded configuration, and FIG. 2B illustrates the expanded member in an expanded configuration. The expandable member 220 may have a length of 4 cm in the unexpanded configuration and may inflate to a diameter between 1 cm and 3 cm. However, the length and inflatable diameter of the expandable member 220 may vary depending on the tissue that the catheter 200 is used on. In other examples, the expandable member 220 may be expanded in other ways (e.g., rolling, unfurling, pushing).

The catheter 200 may further include a plurality of electrodes 230. The plurality of electrodes 230 may be used to provide both electrolysis and electroporation to the targeted tissue. While a plurality of electrodes 230 are shown, in some examples multiple pairs of electrodes may be used. In some examples, one electrode may be disposed on the catheter 200 and may be used in conjunction with a return electrode (e.g., in a unipolar configuration). In some examples, the electrodes may be positioned and/or spaced to create and/or promote a uniform electric field. In some examples, one or more electrodes may be physically connected to one or more other electrodes (e.g., using a web or other portion of connecting material) to create a fixed spacing between the electrodes.

In the illustrated embodiment, the plurality of electrodes 230 extend in a longitudinal direction from at least a proximal portion 222 to a distal portion 224 of the expandable member 220. The number of electrodes 230 may be between two and 30 electrodes, and may be between 12 and 16 electrodes in some examples. Other numbers of electrodes may also be used. In some embodiments, the plurality of electrodes 230 have a round cross-section. In some embodiments, the plurality of electrodes may have a flat surface. In some examples, needle electrodes may be used (e.g., electrodes that wholly and/or partially penetrate tissue). The plurality of electrodes 230 may be alternated between an anode and a cathode arrangement. The anodes and cathodes of the plurality of electrodes 230 may be separated by dielectrics at any point where they intersect. In some embodiments, the anodes and cathodes may crisscross with each other so that in a compressed state the electrodes may be similar in design to a Chinese finger trap.

The anodes and cathodes may be fabricated from any conductive materials such as stainless steel, titanium, graphene, graphite, and the like. Generally, electrode materials may be selected such that the electrode material does not actively participate in electrolysis products and/or leave material residue. For example, the electrode material may be chosen to minimize transferring ions from the electrode material to the target tissue. For example, steel may be less preferred. Titanium and gold may have some degree of participation in the electrolysis process; however, they may not generate a toxic residue and may be used in some examples. In some examples, titanium may be used and may be preferred. In some examples, stainless steel may be used. In some examples, two separate metals such as zinc and aluminum may be used that when left in close proximity produce a current and thus may generate electrolysis products. The diameter of the plurality of electrodes 230 may be around 0.88 mm in some examples, although other sizes may be used. When in the unexpanded configuration, the spacing between the adjacent electrodes 230 may be between 0.05 mm and 0.005 mm in some examples, although other spacings may be used. In the expanded configuration, the spacing between adjacent electrodes 230 may be around 5 mm in some examples, although other spacings may be used.

A distal portion 234 of each electrode 230 may be fixed to a distal end 212 of the elongate member 210 or the distal portion 224 of the expandable member 220. A proximal portion 232 of each electrode 230 may be coupled to a ring 236 that is proximal to the expandable member 220. When the expandable member 220 is inflated or otherwise expanded, the plurality of electrodes 230 are disposed on an outer surface 226 of the expandable member 220. In some examples, the ring 236 may move to accommodate the expansion. In some examples, the ring 236 may remain stationary.

The plurality of electrodes 230 may be attached to two or more wires that run along the elongate member 210 that provide electrical signals, such as voltages and/or currents to the plurality of electrodes 230. These two wires run down the length of the elongate member 210 to the controller 104 and/or power supply 102 generator, which may be outside the body. Once the expandable member 220 has been positioned in place and inflated to the expandable configuration (with the expandable member 220 compressing the stent onto the gastrointestinal wall) a current is run through the electrode causing an electrolysis and electroporation field. The applied electric field can be between 100 V/cm and 1500 V/cm. The expandable member 220 may then be deflated. In some embodiments, the expandable member 220 may be removed and the plurality of electrodes 230 may be left in place to continue producing electrolytic species (e.g., if the plurality of electrodes are made of self-powering materials) to inhibit regrowth of the tissue 10 or other anatomy over an extended period of time, from days to years. For example, one or more electrodes may be made of a piezoelectric and/or thermoelectric material that may generate power over time. In some examples, a power source may be placed in the tissue, such as a battery. In some examples, a microbattery may be used that may include one or more of the electrodes described herein, or other electrodes. The microbattery may in some examples be charged using micro currents in the tissue (e.g., bodily microcurrents). For example, a stent may be used having such a microbattery.

FIG. 3 illustrates a catheter 300 according to examples described herein. The catheter 300 may provide electrolysis and electroporation treatment according to the embodiments of the present disclosure. The catheter 300 includes an elongate member 310 with a distal portion 312 and a proximal portion (not shown). The elongate member 310 may be a tubular member that includes a lumen 314 that extends from the proximal portion (not shown) to the distal portion 312. The distal portion 312 may have a closed end. The catheter 300 may be introduced to a targeted tissue for ablation, delivery of electrolysis products, and regeneration, such as a cavity 12 of the tissue 10 of the small intestine of a patient.

The catheter 300 may further include a plurality of electrodes 330 disposed at a distal portion 312 of the elongate member 310. The plurality of electrodes 330 may be used to provide both electrolysis and electroporation. The plurality of electrodes 330 may include a distal electrode 332 and a proximal electrode 334. Each electrode 332, 334 may be wrapped around the elongate member 310.

The distal portion 312 of the elongate member 310 may include (e.g., may define) a plurality of apertures 316. The plurality of apertures 316 may be disposed between the distal electrode 332 and the proximal electrode 334. An electrically conductive gel 318 may be advanced from the proximal portion of the elongate member 310 to the distal portion 312 of the elongate member 310 through the lumen 314 to exit the elongate member 310 through the plurality of apertures 316. For example, the electrically conductive gel 318 may fill the cavity 12 up to the tissue 10 of the small intestine to aid in shaping the electric field and/or delivery of electrolysis products to the targeted tissue.

FIG. 4 illustrates a schematic of a system 400 according to one embodiment of the present disclosure. The system 400 may include a catheter 410 with an electrode 430 disposed at a distal portion of the catheter 410. The system 400 may also include ground pads 440 that are in operational communication with the electrode 430 of the catheter 410. Accordingly, one or more ground electrodes (e.g., ground pads) may be used as some or all of the electrodes described herein. Generally, the ground electrode may be coupled to a reference voltage (e.g., a ground). While not shown in FIG. 1, note that ground electrodes may also be present in the system described with respect to FIG. 1 and, e.g., placed on or proximate a patient. The ground electrodes may facilitate generation of an electric field in the tissue as described herein.

The system of FIG. 4 may accordingly be used to implement and/or be implemented by the system of FIG. 1. For example, the catheter 200 or the catheter 300 may be used to implement and/or may be implemented by the catheter 410 of FIG. 4. One or more ground electrodes may be provided in the system of FIG. 1 that may additionally be coupled to the catheter 200 or the catheter 300 and/or the power supply 102 or controller 104.

In some embodiments, the ablation instrument delivering electrolytic electroporation may be delivered via a computer-assisted teleoperational manipulator system, sometimes referred to as a robotically assisted system or a robotic system. The manipulator system comprises one or more manipulators that can be operated with the assistance of an electronic controller (e.g., computer) to move and control functions of one or more instruments when coupled to the manipulators.

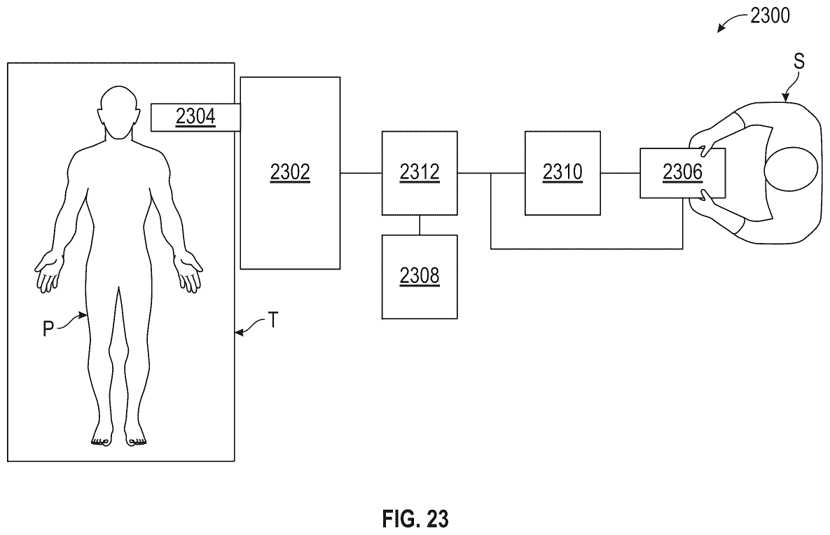

FIG. 23 illustrates an embodiment of a computer-assisted manipulator system for use with the ablation instruments described herein. The manipulator system may be used, for example, in surgical, diagnostic, therapeutic, biopsy, or non-medical procedures (which may collectively be referred to as “surgical” herein), and is generally indicated by the reference numeral 2300. As shown in FIG. 23, a manipulator system 2300 generally includes a teleoperational manipulator assembly 2302 for operating a medical instrument system 2304 in performing various procedures, for example, a medical procedure on a patient P. The medical instrument system 2304 may include one or more steerable instruments and/or one or more passive instruments. One or more instruments of the medical instrument system 2304 may be configured to be positioned within a working channel of one or more other instruments of the medical instrument system 2304. In some examples, one or more surgical instruments or tools may be positionable within one or more working channels of an instrument such as a catheter or endoscope of the medical instrument system 2304. The manipulator assembly 2302 is mounted to or near a patient support table T that may be located in a surgical operating room or other medical setting. An operator input system 2306 allows the operator (e.g., a clinician, surgeon, or other personnel) S to view the interventional site and to control the manipulator assembly 2302. A single manipulator assembly 2302, medical instrument system 2304, and operator input system 2306 is shown in FIG. 23. However, it should be understood that various teleoperated systems may have a plurality of manipulator assemblies, medical instrument systems (each including one or more medical instruments), operator input systems, or combinations thereof.

The operator input system 2306 may be located at a user control system that is usually located in the same room as patient support table T. However, it should be understood that the operator S can be located in a different room or a completely different building or be geographically remote from the patient P. The operator input system 2306 generally includes one or more control devices for controlling the manipulator assemblies 2302. The control devices may include any number of a variety of input devices or sensors, such as joysticks, trackballs, data gloves, trigger-guns, hand-operated controllers, eye tracking devices, voice recognition devices, body motion or presence sensors, or the like. In some embodiments, the control devices will be provided with the same degrees of freedom as one or more associated medical instruments systems (such as the medical instrument system 2304) to provide the operator with telepresence, or the perception that the control devices are integral with the medical instrument systems so that the operator has a sufficiently strong sense of directly controlling the medical instrument systems. In other embodiments, the control devices may have more or fewer or different degrees of freedom than the one or more associated medical instrument systems (such as the medical instrument system 2304) and still provide the operator with telepresence. In some embodiments, the control devices are manual input devices that move with six degrees of freedom, and that may also include an actuatable handle for actuating instruments (for example, for closing grasping jaws, applying an electrical potential to an electrode, delivering a medicinal treatment, or the like).

The teleoperational manipulator assembly 2302 supports the medical instrument system 2304 and may include a kinematic structure of one or more non-servo and/or servo-controlled links (e.g., one or more links that may be manually or robotically positioned and locked in place, generally referred to as a set-up structure) and a teleoperational manipulator. The teleoperational manipulator assembly 2302 includes a plurality of actuators or motors that drive inputs on one or more instruments of the medical instrument system 2304 in response to commands from the control system (e.g., a control system 2312). The motors include drive systems that when coupled to the medical instrument system 2304 may advance the instrument(s) of the medical instrument system 2304 into a naturally or surgically created anatomic orifice. Other motorized drive systems may move the distal end of the instrument(s) of the medical instrument system 2304 in multiple degrees of freedom, which may include three degrees of translational motion (e.g., translational motion along the X, Y, Z Cartesian axes) and in three degrees of rotational motion (e.g., rotation about the X, Y, Z Cartesian axes). Additionally, the motors can be used to actuate an articulable end effector of an instrument for grasping tissue in the jaws of a biopsy device or the like. Motor position or speed sensors such as resolvers, encoders, potentiometers, and other mechanisms may provide sensor data to the teleoperational assembly describing the rotation and orientation of the motor shafts. This position sensor data may be used to determine motion of the objects manipulated by the motors.

The manipulator system 2300 also includes a sensor system 2308 with one or more sub-systems for receiving information about the sub-assemblies of the manipulator system 2300 including instruments of the manipulator assembly 2302. Such sub-systems may include at least one of a position/location sensor system (e.g., an electromagnetic (EM) sensor system); a shape sensor system for determining the position, orientation, speed, velocity, pose, and/or shape of at least a portion of an instrument of the medical instrument system 2304, such as a flexible steerable body or a rigid instrument body with or without joints; a visualization system for capturing images from the distal end of an instrument; other sensor systems based on various sensor technologies; or a combination thereof.

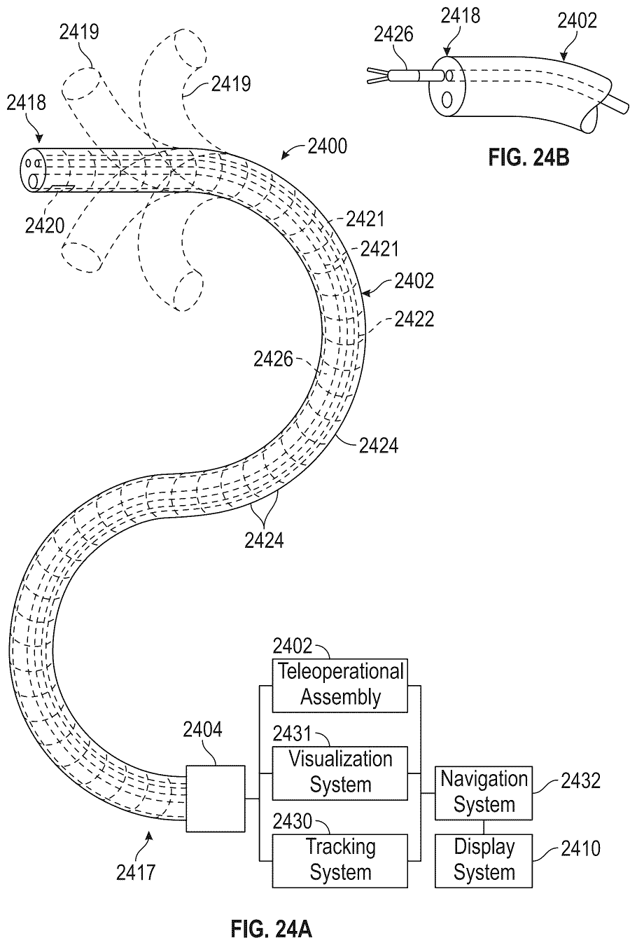

In some examples, a visualization system (e.g., a visualization system 2431 of FIG. 24A) may include a viewing scope assembly that records a concurrent or real-time image of the surgical site and provides the image to the operator (e.g., clinician or surgeon or other personnel) S. The concurrent image may be, for example, a two- or three-dimensional image captured by an endoscope positioned within the surgical site. In some embodiments, the visualization system includes endoscopic components that may be integrally or removably coupled to one or more instruments of the medical instrument system 2304. However, in alternative embodiments, a separate endoscope, attached to a separate manipulator assembly, may be used with the medical instrument system 2304 to image the surgical site. The visualization system may be implemented as hardware, firmware, software, or a combination thereof that interacts with or is otherwise executed by one or more computer processors, which may include the processors of a control system 2312. The processors of the control system 2312 may execute instructions comprising instruction corresponding to processes disclosed herein.

The manipulator system 2300 also includes a display system 2310 for displaying an image or representation of the surgical site and/or medical instrument system(s) 2304 generated by sub-systems of the sensor system 2308. The display 2310 and the operator input system 2306 may be oriented so the operator can control the medical instrument system 2304 and the operator input system 2306 with the perception of telepresence.

The display system 2310 may also display an image of the surgical site and medical instruments (e.g., instruments of medical instrument system 2304) captured by the visualization system. The display 2310 and the control devices may be oriented such that the relative positions of the imaging device in the scope assembly and the medical instruments are similar to the relative positions of the operator's eyes and hands so the operator can manipulate the medical instrument system 2304 and the hand control as if viewing the workspace in substantially true presence. By true presence, it is meant that the presentation of an image is a true perspective image simulating the viewpoint of an operator that is physically manipulating the medical instrument system 2304. Alternatively or additionally, the display 2310 may present images of the surgical site recorded pre-operatively or intra-operatively and/or a virtual navigational image.

The manipulator system 2300 also includes the control system 2312. The control system 2312 includes at least one memory and at least one computer processor (not shown), and in some embodiments typically a plurality of processors, for effecting control between the medical instrument system 2304, the operator input system 2306, the sensor system 2308, and the display system 2310. The control system 2312 also includes programmed instructions (e.g., a computer-readable medium storing the instructions) stored on non-transitory processor readable storage medium to implement some or all of the methods described in accordance with aspects disclosed herein, including instructions for providing information to the display system 2310. While the control system 2312 is shown as a single block in the simplified schematic of FIG. 23, the control system 2312 may include two or more data processing circuits with one portion of the processing optionally being performed on or adjacent the teleoperational manipulator assembly 2302, another portion of the processing being performed at the operator input system 2306, and the like. Any of a wide variety of centralized or distributed data processing architectures may be employed. Similarly, the programmed instructions may be implemented as a number of separate programs or subroutines, or they may be integrated into a number of other aspects of the manipulator systems described herein. In one embodiment, the control system 2312 supports one or more wired or wireless communication protocols. Wireless communications protocols include examples such as Bluetooth, IrDA, HomeRF, IEEE 802.11, DECT, and Wireless Telemetry.

In some embodiments, the control system 2312 may include one or more servo controllers that receive force and/or torque feedback from one or more medical instruments (such as those of the medical instrument system 2304). Responsive to the feedback, the servo controllers transmit signals to the operator input system 2306. The servo controller(s) may also transmit signals instructing the teleoperational assembly 2302 to move a medical instrument that extends into an internal surgical or therapeutic site within the patient body via openings in the body. Any suitable conventional or specialized servo controller may be used. A servo controller may be separate from, or integrated with, the teleoperational assembly 2302. In some embodiments, the servo controller and teleoperational assembly 2302 are provided as part of a teleoperational arm cart configured to be positioned adjacent to the patient's body during a surgical procedure.

The manipulator system 2300 may further include optional operation and support systems (not shown) such as illumination systems, steering control systems, irrigation systems, suction systems, cautery or energy application system, other systems, or combinations thereof. In alternative embodiments, the manipulator system 2300 may include more than one teleoperational assembly 2302 and/or more than one operator input system 2306. The exact number of teleoperational manipulator assemblies will depend on the surgical procedure and the space constraints within the operating room, among other factors. The operator input systems may be collocated or they may be positioned in separate locations that are geographically close or remote from each other. Multiple operator input systems allow more than one operator to control one or more manipulator assemblies in various combinations.

FIG. 24A illustrates a medical instrument system 2400 in accordance aspects of the present disclosure, which may be used as or in the medical instrument system 2304 in an image-guided medical procedure performed with manipulator system 2300. Alternatively, the medical instrument system 2400 may be used for non-teleoperational exploratory procedures or in procedures involving traditional manually operated medical instruments, such as endoscopy.

The instrument system 2400 includes a flexible steerable body 2402 coupled to a housing 2404. The flexible steerable body 2402 has a proximal end 2417 and a distal end or tip portion 2418. In various embodiments, the flexible steerable body 2402 has a size and shape to reach a target anatomy, such as, for example, a 4 mm to 25 mm diameter. Other flexible steerable body outer diameters may be larger or smaller. The instrument system 2400 may optionally include one or more shape sensors 2422 for determining the position, orientation, speed, velocity, pose, shape, or other physical characteristic of the flexible steerable body tip at the distal end 2418, of one or more segments 2424 along the flexible steerable body 2402, and/or along at least a portion of an instrument positionable within channels 2421 of the flexible steerable body 2402 (for example, instrument 2426). The entire length of the flexible steerable body 2402, between the distal end 2418 and the proximal end 2417, may be effectively divided into the segments 2424.

The medical instrument system 2400 (e.g., the flexible steerable body 2402 and/or instruments 2426) may, optionally, include one or more position sensor systems 2420 and/or shape sensors 2422 that may be provided within or mounted externally to the flexible steerable body 2402 or instrument 2426. A tracking system 2430 may include one or more position sensor systems 2420 and one or more shape sensors 2422 for determining the position, orientation, speed, pose, and/or shape of the instruments. The tracking system 2430 may be implemented as hardware, firmware, software, or a combination thereof that interacts with or is otherwise executed by one or more computer processors, which may include the processors of a control system 2312.

The flexible steerable body 2402 includes one or more channels 2421 sized and shaped to receive one or more medical instruments 2426. Medical instruments may include, for example, image capture devices (e.g., an endoscope, such as a monoscopic or stereoscopic endoscope), electrosurgical devices, biopsy instruments, laser ablation fibers, or other surgical, diagnostic, or therapeutic tools. Medical instruments may include end effectors having one or more working members such as a scalpel, a blunt blade, an optical fiber, or an electrode. Other end effectors may include, for example, forceps, grippers, scissors, clip appliers, etc. Examples of electrically activated end effectors include electrosurgical electrodes, transducers, sensors, and the like. One or more of the channels 2421 may have a diameter of approximately 3 mm to 20 mm, for example. In one example, one or more channels 2421 may be configured to receive an approximately 5 mm instrument may have a diameter of approximately 6 mm. Other channels 2421 may have a larger diameter to receive a larger instrument such as an image capture device. For example, the flexible steerable body 2402 may include a channel 2421 sized to receive a larger instrument such as an image capture device, and two lumens each sized to receive a flexible instrument 2426. However, other embodiments may include more or fewer lumens (such as one, two, three, or more lumens).

In various embodiments, one or more of the medical instruments 2426 may be or include an image capture device that includes a distal portion with a stereoscopic or monoscopic camera that are processed by a visualization system 2431 for display. The image capture device may include a cable coupled to the camera for transmitting the captured image data. Alternatively, the image capture device may be a fiber-optic bundle, such as a fiberscope, that couples to the visualization system. The image capture device may be single or multi-spectral, for example capturing image data in one or more of the visible, infrared, or ultraviolet spectrums.

In some embodiments, the flexible steerable body 2402 may include an image capture device, such as a stereoscopic camera, disposed at or near the distal end 2418, for capturing images (including video images). A plurality of lumens 2421 extending through the flexible steerable body 2402 may provide access for a plurality of instruments 2426 to access a surgical site within a field of view of the image capture device.

The medical instrument 2426 may house cables, linkages, or other actuation controls (not shown) that extend between the proximal and distal ends of the instrument to controllably bend the distal end of the instrument. The flexible steerable body 2402 may also houses cables, linkages, or other steering controls (not shown) that extend between the housing 2404 and the distal end 2418 to controllably bend the distal end 2418 as shown, for example, by the broken dashed line depictions 2419 of the distal end of the flexible steerable body 2402. In embodiments in which the instrument system 2400 is actuated by a teleoperational assembly such as the teleoperational assembly 2302, the housing 2404 may include drive inputs that removably couple to and receive power from motorized drive elements of the teleoperational assembly. In embodiments in which the instrument system 2400 is manually operated in whole or in part, the housing 2404 may include gripping features, manual actuators, or other components for manually controlling the motion of the instrument system 2400. The instrument system 2400 may be steerable or, alternatively, the system may be non-steerable with no integrated mechanism for operator control of the instrument bending. Also or alternatively, one or more lumens, through which medical instruments can be deployed and used at a target surgical location, are defined in the walls of the flexible steerable body 2402.

In various embodiments, the medical instrument system 2400 may include a flexible instrument suited for navigation operated by a navigation system 2432 and treatment of tissues, via natural or surgically created connected passageways, in any of a variety of anatomic systems, including the colon, the intestines, the kidneys, the brain, the heart, the circulatory system, pulmonary system, the stomach, other gastrointestinal passageways, and the like.

In the embodiment of FIG. 24A, the instrument system 2400 is teleoperated within the manipulator system 2300 operated by the navigation system 2432 that may provide interfaces through a display system 2410. In an alternative embodiment, the teleoperational assembly 2302 may be replaced by direct operator control. In the direct operation alternative, various handles and operator interfaces may be included for handheld operation of the instrument.

As shown in greater detail in FIG. 24B, medical instruments (such as medical instrument 2426) for such procedures as surgery, biopsy, ablation, illumination, irrigation, or suction can be deployed through one or more channels of the flexible steerable body 2402 and used at a target location within the anatomy. The medical instrument 2426 may be used with an image capture device (e.g., an endoscope) also within the flexible steerable body 2402. Alternatively, the medical instrument 2426 may itself be the image capture device. The medical instrument 2426 may be advanced from the opening of a channel 2421 to perform a procedure and then retracted back into the channel 2421 when the procedure is complete. In some embodiments, optionally, the medical instrument 2426 may be removed from the proximal end 2417 of the flexible steerable body 2402 or from another optional instrument port (not shown) along the flexible steerable body.

Other configurations of teleoperated manipulator systems are also contemplated, such as systems configured for multi-port or single-port procedures. For example, the embodiments described herein may be used with a da Vinci® Surgical System, such as the da Vinci X®, Xi®, or SP® Surgical Systems, all commercialized by Intuitive Surgical, Inc., of Sunnyvale, California.

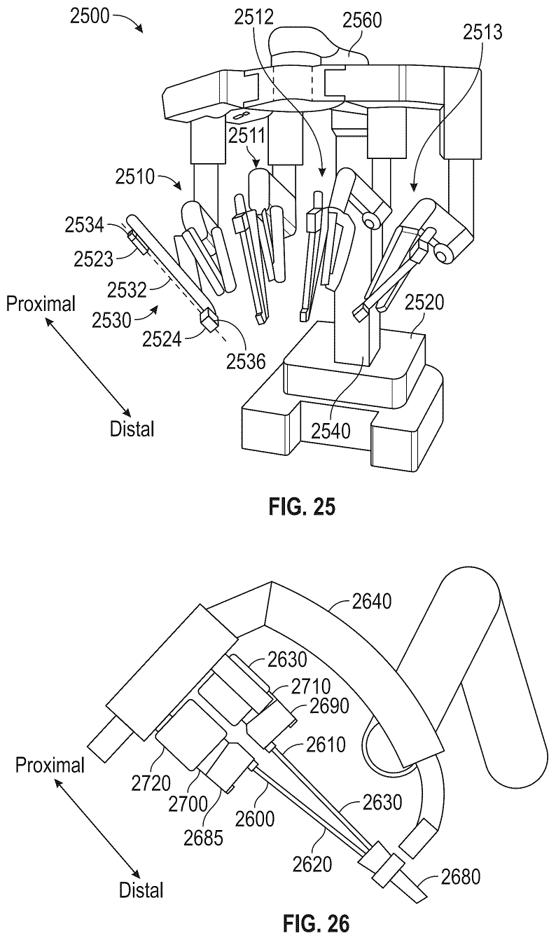

FIG. 25 illustrates an example embodiment of a manipulator system 2500 that may be used as part of the manipulator system 2300. The manipulator system 2500 includes a base 2520, a main column 2540, and a main boom 2560 connected to main column 2540. The manipulator system 2500 also includes a plurality of manipulator arms 2510, 2511, 2512, 2513, which are each connected to main boom 2560. The manipulator arms 2510, 2511, 2512, and 2513 may be used as the manipulator assembly or assemblies 2302. Manipulator arms 2510, 2511, 2512, 2513 each include an instrument mount portion to which an instrument 2530 may be mounted, which is illustrated as being attached to manipulator arm 2510. While the manipulator system 2500 depicts four manipulator arms, various embodiments may include more or fewer manipulator arms.

An instrument mount portion may include a drive assembly 2523 and a cannula mount 2524, with a transmission mechanism 2534 of the instrument 2530 connecting with the drive assembly 2523, according to an embodiment. The cannula mount 2524 is configured to hold a cannula 2536 through which a shaft 2532 of the instrument 2530 may extend to a surgery site during a surgical procedure. The drive assembly 2523 contains a variety of drive and other mechanisms that are controlled to respond to input commands at the operator input system 2306 and transmit forces to the transmission mechanism 2534 to actuate the instrument 2530. Although the embodiment of FIG. 25 shows an instrument 2530 attached to only manipulator arm 2510 for ease of viewing, an instrument may be attached to any and each of manipulator arms 2510, 2511, 2512, 2513.

With reference now to FIG. 26, a portion of an embodiment of a manipulator arm 2640 of a manipulator system with two surgical instruments 2600, 2610 in an installed position is shown. The schematic illustration of FIG. 26 depicts only two surgical instruments for simplicity, but more than two surgical instruments may be mounted in an installed position at a manipulator system as those having ordinary skill in the art are familiar. Each surgical instrument 2600, 2610 includes a shaft 2620, 2630 having at a distal end a moveable end effector or an endoscope, camera, or other sensing device, and may or may not include a wrist mechanism (not shown) to control the movement of the distal end.

In the embodiment of FIG. 26, the distal end portions of the surgical instruments 2600, 2610 are received through a single-port structure 2680 to be introduced into the patient. As shown, the port structure includes a cannula and an instrument entry guide inserted into the cannula. Individual instruments are inserted into the entry guide to reach a surgical site.

Transmission mechanisms 2685, 2690 are disposed at a proximal end of each shaft 2620, 2630 and connect through a sterile adaptor 2700, 2710 with drive assemblies 2720, 2730, which contain a variety of internal mechanisms (not shown) that are controlled by a controller (e.g., at a control cart of a surgical system) to respond to input commands at a surgeon side console of a surgical system to transmit forces to the force the transmission mechanisms 2685, 2690 to actuate the surgical instruments 2600, 2610.

The manipulator and instrument systems 2300, 2400 and 2500 and instruments 2426, 2530, and 2600 are examples of delivery devices that may be used to deliver electrolytic electroporation described herein. For example, the instruments 2426, 2530, and 2600 may include electrode end effectors (e.g., optionally with expandable members) as described herein to produce and deliver electrolytic electroporation to target tissue. The manipulator systems described herein are not limited to the embodiments of FIGS. 23, 24A-24B, 25, and 26, and various other teleoperated, computer-assisted manipulator configurations may be used with the embodiments described herein. The diameter or diameters of an instrument shaft and end effector are generally selected according to the size of the cannula with which the instrument will be used and depending on the surgical procedures being performed.

The disclosed catheters and systems may be used in a variety of different tissue locations. Tissue locations that are known for rapid tissue regeneration may be good options for electrolytic electroporation tissue regeneration. For example, the catheters and systems may be used in the small intestine, kidney, heart, and other types of tissue.

### EXAMPLES

The small intestine was chosen as a model to demonstrate the ability of electrolytic electroporation to precisely ablate a controlled volume such that electrolytic electroporation ablation does not impair the tissue regeneration process.

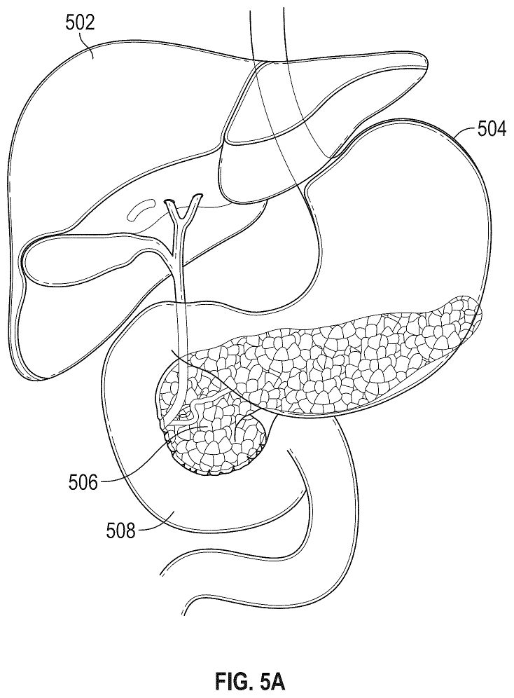

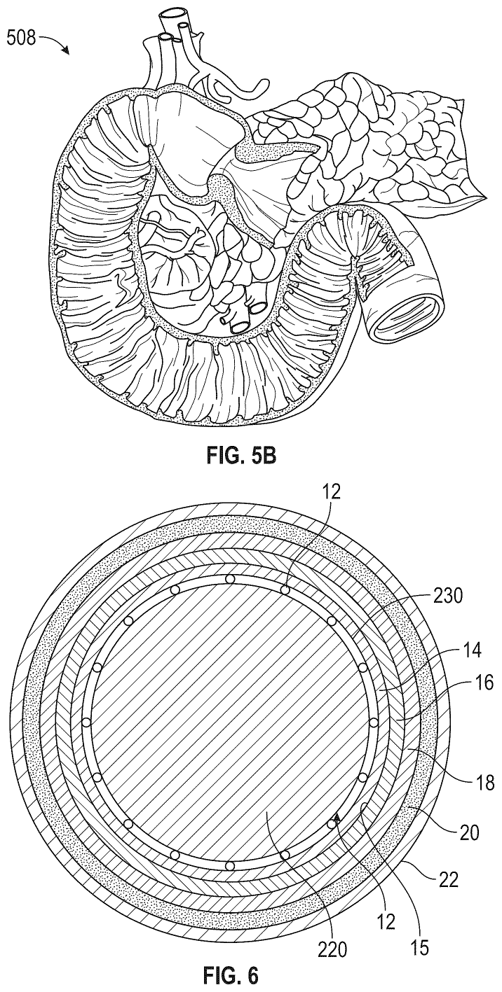

FIG. 5A is a schematic illustration of anatomy arranged in accordance with examples described herein. The anatomy includes a liver 502, stomach 504, pancreas 506, and duodenum 508. Examples of delivery systems described herein may deliver electric fields, and therefore electroporation and electrolysis, to the duodenum 508.

FIG. 5B is a schematic illustration of anatomy arranged in accordance with examples described herein. FIG. 5B is a more detailed view of anatomical feature of the duodenum 508.

The examples are provided to show examples of parameters of electroporation in combination with electrolysis that allow tissue regeneration and to show that electroporation and electrolysis may be delivered precisely.

Minimally invasive regenerative surgery using electrolytic electroporation was performed in the duodenum and jejunum mucosa as an example for the controlled use of electrolytic electroporation for a mode of ablation that facilitates the regeneration of the ablated tissue.

The goal of the examples was to verify that electrolytic electroporation can ablate a desired volume of tissue with precision and to verify that electrolytic electroporation ablation may be used in a way that does not prevent the ability of the tissue in the ablated volume to regenerate. To explore these aspects of electrolytic electroporation ablation, the duodenal mucosa in a pig was used as an experimental model. The duodenum mucosa generally has the ability to regenerate from damage within three to seven days.

The examples included several parts. A mathematical model of the electric fields around the electrodes was developed. The model was used to calculate the voltages on the electrode in one of the electrolytic electroporation catheters or systems that generate reversible electroporation electric fields in the mucosa of the duodenum. As discussed above, and shown in FIG. 1, the catheters or systems were connected to a power supply that can generate a precise voltage and deliver a predetermined charge through the electrodes.

A series of acute studies were performed to evaluate the ability of the electrolytic electroporation technology to ablate to a penetration depth of the deep mucosa only, without causing coagulative necrosis within the underlying muscle layer, e.g., muscularis layer containing the visceral muscle cells, and to study the effect of various treatment parameters within the mathematically calculated range, on the extent of ablation. This was followed by a study in which the electrolytic electroporation parameters for the mucosal ablation that were determined in the previous part were used. The outcome of this treatment was followed with histology taken over time for up to eight days after the electrolytic electroporation treatment, to verify that tissue ablated with electrolytic electroporation has the ability to regenerate. The examples used the discharge of a capacitance in the range of voltages from 100 V to 1000V and a capacitance from 50 μF to 3500 μF across a resistance of 1500 ohm in series with the tissue resistance, between pairs of electrodes.

### Mathematical Model I

A mathematical model was developed to evaluate the electric fields around the expandable member 220 of the embodiment illustrated in FIGS. 2A and 2B. This mathematical model may be used to determine electric fields for use in other examples of the application of electrolysis and electroporation described herein. FIG. 6 illustrates the tissue 10 of the small intestine with the expandable member 220 of the catheter 200 disposed within the cavity 12 of the tissue 10 of the small intestine. The expandable member 220 and the electrodes 230 disposed on the outer surface 226 of the expandable member 220 are pressed against the mucosa 14 along the axis of the duodenum. The illustrated embodiment utilized 16 electrodes that were equally spaced around the outer surface 226 of the expandable member 220. However, as discussed above, the number of electrodes may range between two and 30 electrodes, or some other number of electrodes may be used. FIG. 6 also illustrates the various layers of tissue of the small intestine 10. The layers of tissue 10 of the small intestine include the mucosa 14, the submucosa 16, the inner circular muscularis 18, the outer longitudinal muscularis 20, and the serosal surface 22. An interface 15 between the mucosa and the submucosa 16 is about 0.85 mm from the outer surface 226 of the expandable member 220 (e.g., the depth of the mucosal layer is 0.85 mm). The reference numerals for these tissue features are retained throughout FIGS. 8-20.

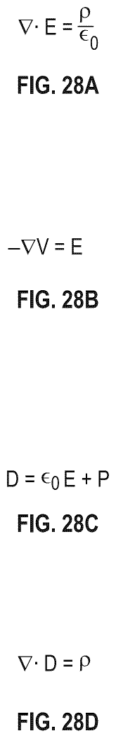

Electric fields produced by the electrodes 230 can be determined by solving the Maxwell equations on a two-dimensional cross-section finite element model of the expandable member 220, the plurality of electrodes 230, and the surrounding tissue of the small intestine 10. The model simulates the electric field around a cross-section in the expandable member 220 in a plane normal to the axis of the duodenum of the small intestine 10. COMSOL Multiphysics (version 5.5) was used for the analysis. The equation for the electric field, E for a free space, with a space charge density p is represented in FIG. 28A, where €, is the absolute permittivity. The electric field is linked to the potential field V, through the relationship represented as an equation of FIG. 28B. The electric displacement field D is defined as an equation in FIG. 28C, where P is the polarization vector field. With the above definition, the equation (also called Gauss's law) is represented in FIG. 28D.

### Example 1—Mathematical Model

The analysis simulates an experiment in which the expandable member 220 was inflated to a diameter of 30 mm. The electric fields decrease from the electrode surface outward, away from the catheter. For safety and so as not to affect the muscularis beyond the submucosa, the electric field was set to be at a level that yields reversible electroporation at a distance of about 0.85 mm from the balloon outer surface 226, e.g., at the outer margin of the mucosa. Since the electric field decreases from the catheter outward, the strength of the electric field beyond the mucosa into the submucosa and the muscularis is below the level required for cell membrane permeabilization. Accordingly, examples described herein (e.g., the system of FIG. 1) may generate an electric field in tissue at a location where the field will permeabilize cells desired for ablation, but may not affect other cells for which ablation is not desired. For example, the permeabilization electric field may be provided at a depth to affect (e.g., permeabilize) the mucosa and/or submucosa, but not the underlying muscle layer. Note that it may be acceptable to cause some permeabilization and/or ablation of the submucosa using reversible electroporation and electrolysis as described herein, in part because the submucosa may regenerate and heal damage that may be caused while ablating the mucosal layer. In the model, a thickness of 0.85 mm was used for the mucosa 14 and a thickness of 2.15 mm was used for the muscle layer 18, 20. In the calculations of the electric field, the relative permittivity for the muscle 18, 20 was taken to be 66 and was 44.79 for the mucosa.

The mathematical model was used to determine the electric field at the interface 15 between the mucosa 14 and the submucosa 16. Specifically, the mathematical model was applied to (a) a 0.5 mm electrode disposed symmetrically around the balloon and (b) a flat electrode attached to the balloon so that the surface of the electrodes 230 and the outer surface 226 of the expandable member 220 adjacent to the electrodes 230 are equal, and the electrodes 230 are also symmetrically disposed around the outer surface 226 of the expandable member 220.

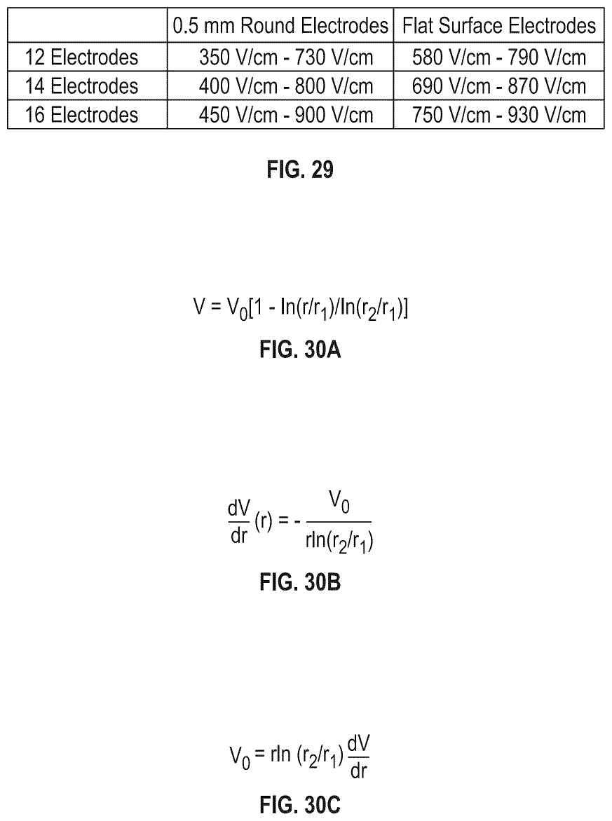

The calculations were performed with a voltage of 500 V between adjacent electrodes 230. However, the electric fields scale linearly based on the applied voltage. For example, an applied voltage of 1000 V will generate an electric field that is twice as big as that generated by an applied voltage of 500 V, and an electric field generated by an applied voltage of 250 V is half of that generated by an applied voltage of 500 V. The analysis was performed for 12, 14, and 16 electrodes. The range of electric fields at 0.85 mm from the outer surface 226 of the expandable member 220 for both round electrodes and surface electrodes is summarized in a table in FIG. 29, for different numbers of electrodes. Flat electrodes yield a somewhat higher electric field and the range is narrower.

It is evident that the larger the number of electrodes 230, the higher the electric fields. Furthermore, the maximum electric fields at 0.85 mm are well below the electric fields used in irreversible electroporation. Because the electric fields decay with distance from the electrodes and catheter surface, these electric fields cannot yield irreversible electroporation, and any cells in this range are only affected by the mechanism of ablation with the combination of reversible electroporation and electrolysis.

### Example 2—Mathematical Model

Example 2 is directed to the catheter 300 illustrated in FIG. 3. As discussed above, catheter 300 includes two axial electrodes 332, 334 in which the space between the catheter and the intestine is filled with an electrically conductive gel 318. Electric fields generated by different sized electrodes and spaces between the electrodes may be simulated to identify an electrode size, spacing, and voltage application to result in a field strength at the mucosal layer sufficient to cause reversible electroporation.

### Example 3—Mathematical Model

Example 3 is directed to the system 400 illustrated in FIG. 4. In the mathematical analysis, the system 400 is simulated as a single polarity cylindrical electrode 430 in the intestine 10 and a ground pad on the outer surface of the body. A relationship between the potential V around a cylindrical catheter 410 with a radius r₁ and potential of Vo around a ground pad 440 on the outer surface r₂ is represented in an equation of FIG. 30A. The field is computed as an equation of FIG. 30B. Thus, the potential Vo may be computed as an equation of FIG. 30C.

Assuming r₁=1.75 cm and r₂=30 cm, for r=2 cm and electric field of 300 V/cm at 2 cm we get: V₀=1704 V and the electric field on the electrode at r₁ is 341 V/cm.

Assuming r₁=1.75 cm and r₂=10 cm, for r=2 cm and electric field of 300 V/cm we get: Vₒ=1045 V and the electric field at the electrode at r₁ is 342 V/cm.

### Example 1—Animal Model

In the animal model, a female, 67.3 kg Yorkshire pig was used. A 24-hour study was used for adequate assessment of the ablation sites and effect at the cellular level within the layers of the intestinal wall. In addition to identifying parameters that can achieve mucosal depth ablation, the animal study established that electrolytic electroporation could achieve precise ablation depth without the need for mucosal layer isolation. FIG. 6 is a schematic that shows the small intestines and the mucosa 14 targeted for ablation.

The animals were fasted for 24 hours prior to the procedure (n=2; 60-65 kg). ChloraPrep™ alcohol-based CHG (BD, Vernon Hills, IL) was used on the abdomen as a preoperative skin preparation agent. The animals were prepped and draped in the usual sterile fashion. Preoperative antibiotics (broad-based cephalosporin) were administered prior to the incision.

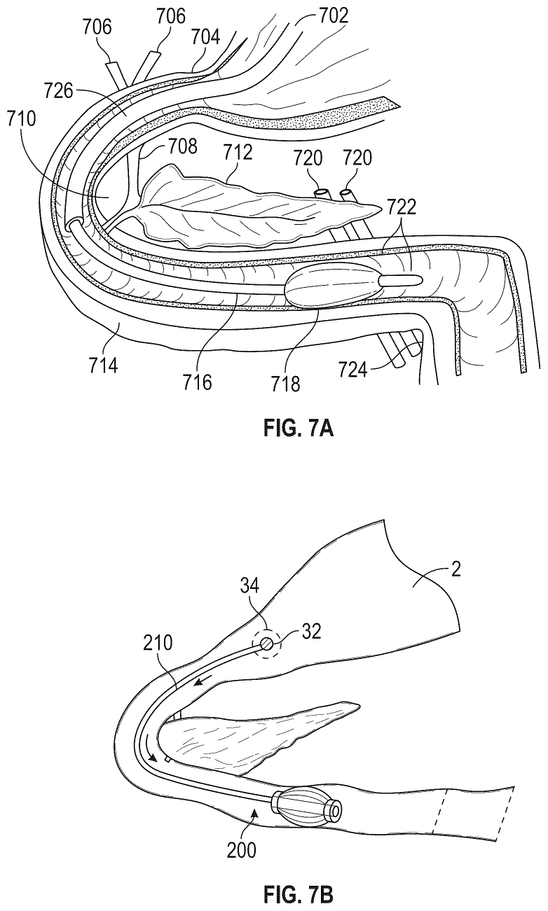

FIGS. 7A and 7B illustrate schematics of a medical procedure for inserting electrolytic electroporation catheters or systems.

FIG. 7A illustrates delivery of a delivery system, a catheter in the example shown, to a duodenum for ablation of mucosal lining in accordance with examples described herein. FIG. 7A illustrates a duodenum 714 in its anatomical environment. Also shown in FIG. 7A are a stomach 702, a pylorus 704, bile ducts 706, a bile duct 708, a nerve plexus 710, a pancreas 712, superior mesenteric vessels 720, a mucosal lining 722, and a ligament of Treitz 724. An endoscope 726 may be delivered in a trans-oral manner to duodenum 714. Accordingly, the endoscope 726 may be delivered through an esophagus and/or stomach into the duodenum 714. A catheter may be delivered by the endoscope 726, such as by being delivered in a working channel of the endoscope 726. The endoscope 726 may include other components, including but not limited to one or more cameras, light source(s), and/or other sensor(s). A shaft 716 of the catheter may be positioned in the duodenum 714. An expandable member 718 may be coupled to the shaft 716 and may expand in a treatment area. The expandable member 718 may include electrodes, such as a linear array of electrodes, for application of electric fields as described herein. In this manner, the catheter may be used to treat a mucosal lining 722. The area to be ablated may be around the expandable member 718, and may be targeted to ablate the mucosal lining 722 without affecting adjacent tissues in some examples.

While the example of FIG. 7A illustrates the use of a delivery system including an endoscope that may be used to deliver a catheter to the treatment area, other delivery systems may additionally or instead be used. For example, the delivery system may be implemented using a computer-assisted teleoperational manipulator system, sometimes referred to as a robotically assisted system or a robotic system. For example, the robotic system shown and described with reference to FIGS. 23-26 may be used to control and/or deliver a catheter through an endoscope described with reference to the robotic system. In some examples, one or more electrodes may be provided as one of the instruments described with reference to the robotic control system of FIGS. 23-26.

FIG. 7B illustrates the catheter 200 being inserted into the small intestine 10 using a surgical hole created for experimental access; however, the present disclosure is not limited thereto or thereby and any catheters or systems discussed above may be used. The catheter 200 is inserted into the gastrostomy 32. A purse-string suture 34 was placed around the gastrostomy 32 in order to aid with securing the catheter 200. The catheter 200 was prepped with Envirocide™ (Metrex Research, Orange, CA) to decontaminate the catheter 200.

The catheter 200 was then manually advanced into the mid portion of the jejunum. While the target was primarily the duodenum for the safety study, the jejunum was also utilized in the study in order to maximize the number of ablation sites in each animal given that the duodenum measured roughly 8 to 10 cm in length allowing for only three separate non-overlapping ablation sites. Electrolytic electroporation duodenal mucosal regeneration (DMRe) was then performed from the mid jejunum back toward the proximal duodenum, working distal to proximal. Once in the proper position, the expandable member 220 of the catheter 200 was insufflated under direct visualization to ensure circumferential tissue apposition. Electrolytic electroporation pulses were then delivered to the tissue (range: 100 V to 1000 V, one to 10 pulses). Fifteen lesions were created in each animal for a total of 30 electrolytic electroporation ablation sites. Very light muscle fasciculations were observed with the pulsed ablations; however, no severe muscle contractions or plasma arcing was observed with the electrolytic electroporation ranges delivered to achieve DMRe. Following each electrolytic electroporation ablation, the lesions were marked directly in the middle of each ablation zone for identification with large metallic vascular clips (Week™ Horizon Clips™, Teleflex, Morrisville, NC) on the surface of the mesentery. Attention was paid so as not to ligate any feeding mesenteric arterial branches that could potentially compromise blood flow to the bowel. The catheter 200 was compressed (e.g., deflated) and then retracted manually using tactile feel in order to space out electrolytic electroporation ablation lesions throughout the small intestine 10. Active electrolysis could be grossly seen on the serosal surface 22 of the bowel wall with a temporary grayish discoloration of the tissue. Fifteen total ablations were performed. Upon completion of the 15 electrolytic electroporation ablations in each animal, the catheter 200 was removed and the gastrostomy 32 was repaired. The tissue 10 of the small intestine was visually inspected and no compromised tissue at risk for necrosis was observed on the serosal surface 22 of the proximal small intestine in either animal. The midline laparotomy 30 was then closed. Each animal was then observed for 24 hours and tolerated a clear diet. No adverse events were observed.

Euthanasia was then performed at 24 hours for each animal. The proximal intestine was dissected with pancreatic and mesenteric sections attached. Each ablation site was then explanted and fixed in formalin. Histological analysis (gross, H&E) was then performed to assess the tissue viability and electrolytic electroporation ablation depth in order to identify parameters to achieve trans-mucosal ablation without causing full-thickness injury to the bowel.

FIGS. 8-11 are photomicrographs of the small intestinal section ablated with the electrolytic electroporation power supply and the catheter 200 and the exponential decay wave generator power supply 102. FIGS. 8-11 are images shown stained with H&E. The lesion boundaries, e.g., the width and depth of treatment penetration, are enclosed within the dark circles or squares where applicable. The ablated effect is variable wherein the lesions are either punctate, multifocal and/or focally extensive involving the mucosa and submucosa, and/or traversed through the full intestinal wall thickness. The multifocal lesions are usually interrupted by wedges of normal mucosa circumferentially. In instances of low degree of tissue effect, the injuries are localized in the mucosa with necrosis and sloughing of the upper half of the villi (resembling ulceration) while the lower half of the villi have lining epithelial cells which appear somewhat like “ghost cell” maintaining architectural outlines. These ghost cells are considered essentially nonviable. The necrosis is accompanied by congestion and/or hemorrhage and infiltration of polymorphonuclear leukocytes, mostly neutrophils. In more severely affected cases, ablation effect is relatively more pronounced with complete destruction of the mucosa and submucosa with or without involvement of the outer muscular layers of the serosa. Intestinal wall perforation is not seen at any given site.

FIG. 8 illustrates a cross-section normal to the axis of the tissue 10 of the intestine and higher magnification of a particular ablation site (encircled). Electrolytic electroporation was applied with a voltage of 500 V, a capacitance of 100 μF, a resistance of 15-20 ohms, one pulse, and time constant of 1.8 ms. The results of the electrolytic electroporation were mucosal ablation and superficial and deep mucosal ablation, the submucosa and muscle remained intact, the villous tips were ablated, there was minimal edema in the submucosal, and ablation was contained within the mucosa. There was a 90% mucosal ablation.

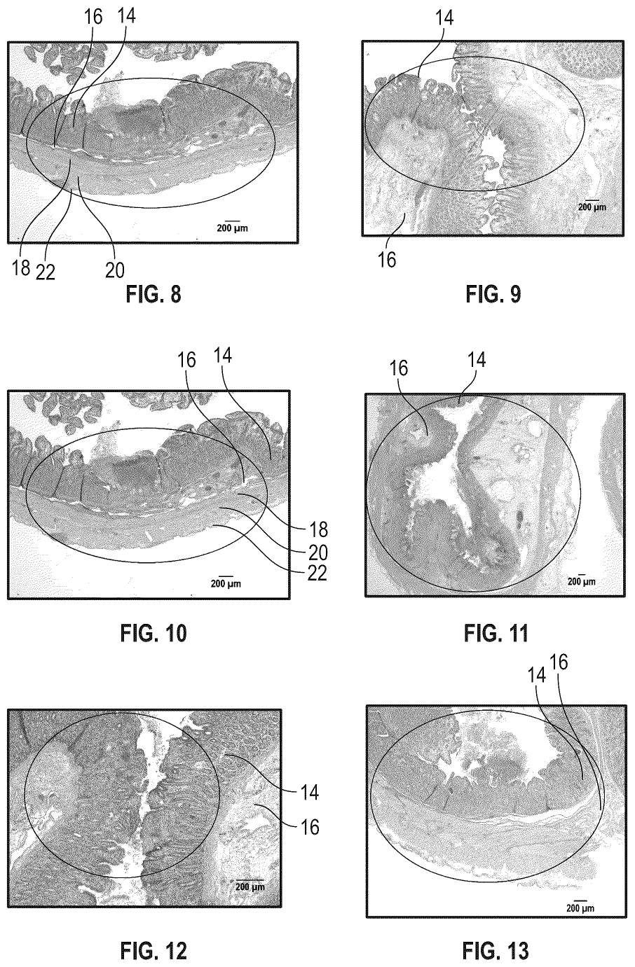

FIG. 9 illustrates a cross-section normal to the axis of the tissue 10 of the intestine and higher magnification of a particular ablation site (encircled). Electrolytic electroporation was applied with a voltage of 500 V, a capacitance of 100 μF, a resistance of 15-20 ohms, two pulses, and a time constant of 1.7 ms. The time interval between the delivery of the pulses was about one minute. The results of the electrolytic electroporation were 75% overall mucosal ablation with the submucosa and muscle intact. The scaffolding of the tissue 10 of the intestine was well preserved.

FIG. 10 illustrates a cross-section normal to the axis of the tissue 10 of the intestine and higher magnification of a particular ablation site (encircled). Electrolytic electroporation was applied with a voltage of 500 V, a capacitance of 100 μF, a resistance of 15-20 ohms, five pulses, and a time constant of 1.7 ms. The time interval between the delivery of the pulses was on the order of one minute or less. The results of the electrolytic electroporation were 100% of overall mucosa and submucosa ablation with the muscularis intact.

FIG. 11 illustrates a cross-section normal to the axis of the tissue 10 of the intestine and higher magnification of a particular ablation site (encircled). Electrolytic electroporation was applied with a voltage of 500 V, a capacitance of 100 μF, a resistance of 15-20 ohms, 10 pulses, and a time constant of 1.7 ms. The time interval between the delivery of the pulses was on the order of one minute or less. The results of the electrolytic electroporation were muscularis intact, ablation within the mucosa and the deep mucosa, some submucosa ablation, and 90% of overall mucosa ablation.

The electrolytic electroporation of FIGS. 8-11 was generated with 16, 0.88 mm diameter round electrodes 230. The calculated electric fields at the interface 15 between the submucosa 16 and the mucosa 14 were calculated to be between 850 V/cm and 450 V/cm, which is in the range of reversible electroporation. Between the outer surface of the catheter and the interface 15, the electric fields do not exceed the range of reversible electroporation electric fields of about 1000V/cm to 1200 V/cm. In some electrode configurations with sharp edges there can be some local points of singularity with irreversible electroporation electric fields. The maximum temperature modeled for the energy applied was 45.88° C. at 0.5 ms at the surface of the electrode. The surrounding temperatures were below the 40° C. FIGS. 5-8 show that the extent of ablation is confined by the extent of the electric field that induces electroporation and does not extend beyond, no matter the number of pulses.

FIG. 12 illustrates an outcome of an experiment, performed with the catheter 200 and a power supply used to generate the results in FIGS. 8-11. However, the voltage was elevated to 750 V and the capacitance to 50 μF, e.g., the amount of products of electrolysis was halved. The electric fields at the interface 15 between the mucosa 14 and the submucosa 16 are between 1125 V/cm and 675 V/cm. The overall parameters were a voltage of 750 V, a capacitance of 50 μF, a resistance of 15-20 ohms, one pulse, and a time constant of 0.529 seconds. The histology shows that there is superficial ablation contained to the mucosa 14. There was a total of 25 to 30% mucosal ablation. The results of the electrolytic electroporation show that despite the elevation in electric fields, a reduction in the products of electrolysis reduces the extent of ablation.

FIG. 13 illustrates the outcome of an experiment performed with the catheter 200 and the power supply 102 used to generate the results in FIGS. 8-11. However, the voltage was elevated to 1000 V and the capacitance to 50 μF, e.g., the amount of products of electrolysis was halved. The electric fields at the interface 15 between the mucosa 14 and the submucosa 16 are between 1700 V/cm and 900 V/cm. The overall parameters were a voltage of 100 V, a capacitance of 50 μF, a resistance of 15-20 ohms, one pulse, and a time constant of 0.532 seconds. The treatment was associated with arcing, which is typical of irreversible electroporation parameters. Indeed 1700 V/cm is within the range of irreversible electroporation parameters. This suggests that the entire mucosa 14 from the electrodes to the submucosa 16 were exposed to irreversible electroporation parameters. (It should be noted that in delivering reversible electroporation it is anticipated that some irreversible electroporation may occur near the electrodes.) The result of the electrolytic electroporation was 25% transmural ablation. This experiment illustrates the possible problems with irreversible electroporation ablation in the intestine 10. For example, irreversible electroporation may damage the cells such that scar tissue is created or other obstacles to tissue regeneration are presented.

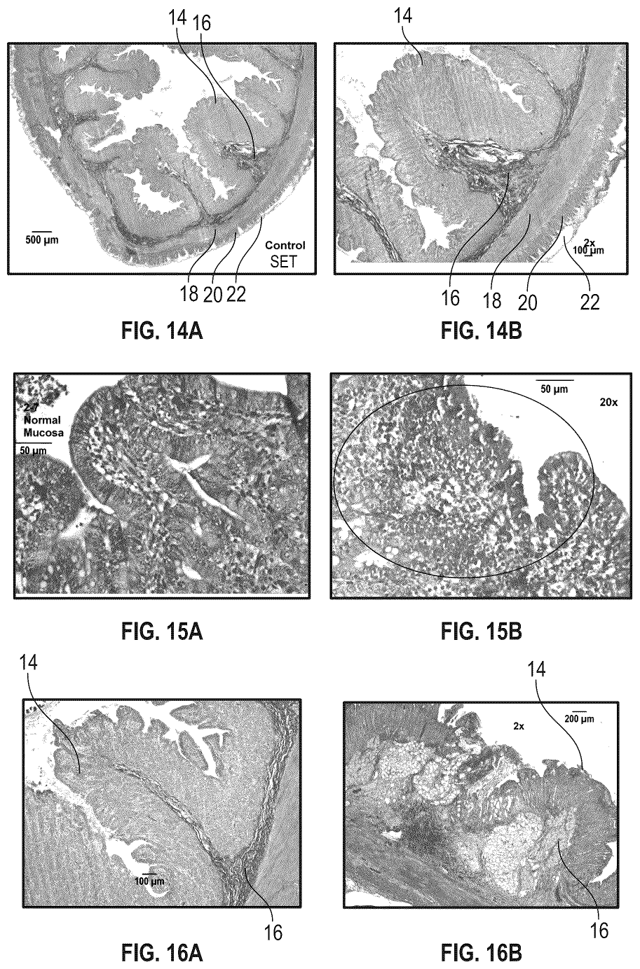

FIGS. 14A and 14B illustrate the outcome of an experiment performed with the catheter 200 and the power supply used to generate the results in FIGS. 8-11. FIG. 14B is a magnified image of FIG. 14A. However, the voltage was kept at 500 V and the capacitance was increased to 250 μF, e.g., the amount of products of electrolysis is 2.5 times that used in experiments in FIGS. 8-11. The electric fields at the margin of the mucosa are the same as in FIGS. 8-11, between 450 V/cm and 850 V/cm. The overall parameters were a voltage of 500 V, a capacitance of 250 μF, a resistance of 15-20 ohms, one pulse, and a time constant of 3.6 ms. The histology shows areas of transmural ablation, ulcerated and coagulated submucosa, and muscle. In general, there was 20% ablation and 10% transmural ablation. This experiment shows the potential effect of the instantaneous delivery of a large amount of products of electrolysis.

The set of experiments depicted in FIGS. 8-14 show that (1) it is possible to design an electrolytic electroporation treatment protocol that ablates precisely the targeted tissue, (2) increasing the electric field to the irreversible electroporation range can cause undesirable damage, and (3) increasing the amount of products of electrolysis can also result in undesirable damage. The conclusion is that electrolytic electroporation can produce a desired mode of ablation; however, it must be designed and delivered precisely for the application intent. This demonstrates that the use of electrolytic electroporation for precise ablation and subsequent regeneration is a surprising and unexpected result of combining electrolysis and electroporation. For example, parameters may be used which result in the extracellular matrix of ablated tissues remaining intact, facilitating tissue regeneration.

### Tissue Regeneration Study at 24 Hours, Three Days, and Eight Days

A porcine model was chosen due to its analogous structure to that of humans in terms of diameter and bowel wall thickness.

A prior 24-hour porcine study identified electrolytic electroporation parameters that achieved trans-mucosal ablation down to the level of the submucosa without disrupting or causing necrosis within the muscularis. This established proof of concept that judiciously delivered electrolytic electroporation can achieve precise mucosal depth ablation. This demonstrated duodenal mucosa regeneration after treatment with the electrolytic electroporation parameters identified in the previous series of experiments. In a series of three pig experiments, the regeneration process in animals treated with the parameters listed in the table of FIG. 29 were evaluated. The catheter 200 and the power supply were the same as in the previous experiments.

The animals were fasted for 24 hours prior to the procedure (n=3; 60-65 kg). ChloraPrep™ alcohol-based CHG (BD, Vernon Hills, IL) was used on the abdomen as a preoperative skin preparation agent. The animals were prepped and draped in the usual sterile fashion. Preoperative antibiotics (broad-based cephalosporin) were administered prior to the incision.

As discussed above, FIGS. 7A and 7B illustrate a schematic of a medical procedure for inserting electrolytic electroporation electrode catheters or systems. FIG. 7A illustrates a midline laparotomy and the exteriorization of the small intestine 10. In some embodiments, the stomach 2 may also be exteriorized. The gastrostomy 32 for surgical access was created in order to manually advance the electrolytic electroporation catheters or systems into the proximal small intestine 10.

FIG. 7B illustrates the catheter 200 being inserted into the tissue 10 of the small intestine through the gastrostomy 32. A purse-string suture 34 was placed around the gastrostomy in order to aid with securing the catheter 200. The catheter 200 was prepped with Envirocide™ (Metrex Research, Orange, CA) to decontaminate the device.

The catheter 200 was then manually advanced into the mid portion of the jejunum for each animal. While the target was primarily the duodenum for the safety study, the jejunum was also utilized in the study to maximize the number of ablation sites in each animal given that the length of the duodenum measured roughly 8 to 10 cm allowing for only three separate non-overlapping ablation sites. Electrolytic electroporation DMRe was then performed from the mid jejunum back toward the proximal duodenum, working distal to proximal. Once in the proper position, the expandable member 220 of the catheter 200 was then insufflated under direct visualization to ensure circumferential tissue apposition. Electrolytic electroporation pulses were then delivered to the tissue (Range: 500 V, one, two, and five pulses). Twelve ablations were performed in each animal for 36 total ablations. Three lesions with one pulse, three lesions with two pulses, and five lesions with five pulses were observed in each animal. The table in FIG. 29 is a list of the experiments performed in this study.

Very light muscle fasciculations were observed with the pulsed ablations; however, no severe muscle contracts or plasma arcing was observed with the electrolytic electroporation ranges delivered to achieve DMRe. Following each electrolytic electroporation ablation, the lesions were marked directly in the middle of each ablation zone for identification with large metallic vascular clips (Week™ Horizon Clips™ Teleflex, Morrisville, NC) on the surface of the mesentery. Attention was paid so as not to ligate any feeding mesenteric arterial branches that could potentially compromise blood flow to the bowel. The catheter 200 was contracted (e.g., deflated) and then retracted manually using tactile feel in order to space out electrolytic electroporation ablation lesions throughout the small intestine 10. Active electrolysis could be grossly seen on the serosal surface 22 of the bowel wall with a temporary grayish discoloration of the tissue. Fifteen total ablations were performed. Upon completion of the electrolytic electroporation ablations, the catheter 200 was removed and the gastrostomy 32 was repaired. The tissue 10 of the small intestine was visually inspected and no compromised tissue at risk for necrosis was observed on the serosal surface 22 of the proximal intestine 10. The midline laparotomy 30 was then closed. Each animal was then returned to a monitoring area and tolerated a clear diet. No adverse events were observed in the post-procedural period for any of the animals.

Euthanasia was then performed at 24 hours, three days, and eight days. The proximal intestine was dissected with pancreatic and mesenteric sections attached. Each ablation site was then explanted and fixed in formalin. Histological analysis (gross, H&E) was then performed to assess the tissue viability and electrolytic electroporation ablation depth in order to identify parameters to achieve trans-mucosal ablation without causing full-thickness injury to the bowel. No bowel necrosis, ulcerations, strictures, or perforations or were observed in any of the electrolytic electroporation ablation zones during gross visualization and inspection at the time of explant.

While tissue regeneration is shown out through eight days in these examples, tissue regeneration may occur, in some examples without scar or fibrosis (e.g., with the absence of collagen bands) out through longer periods of time including 10 days, 20 days, or 30 days in some examples.

FIG. 15A illustrates mucosa of a control sample. FIG. 15B illustrates the appearance of the mucosa 24 hours after the treatment of the mucosa with one single electrolytic electroporation pulse of 500 V and 100 μF.

FIG. 16A illustrates mucosa of a control sample. FIG. 16B illustrates the appearance of the mucosa 24 hours after the treatment with two electrolytic electroporation pulses of 500 V and 100 μF. Two pulses appear to cause more substantial ablation of the mucosa; however, the muscularis is intact.

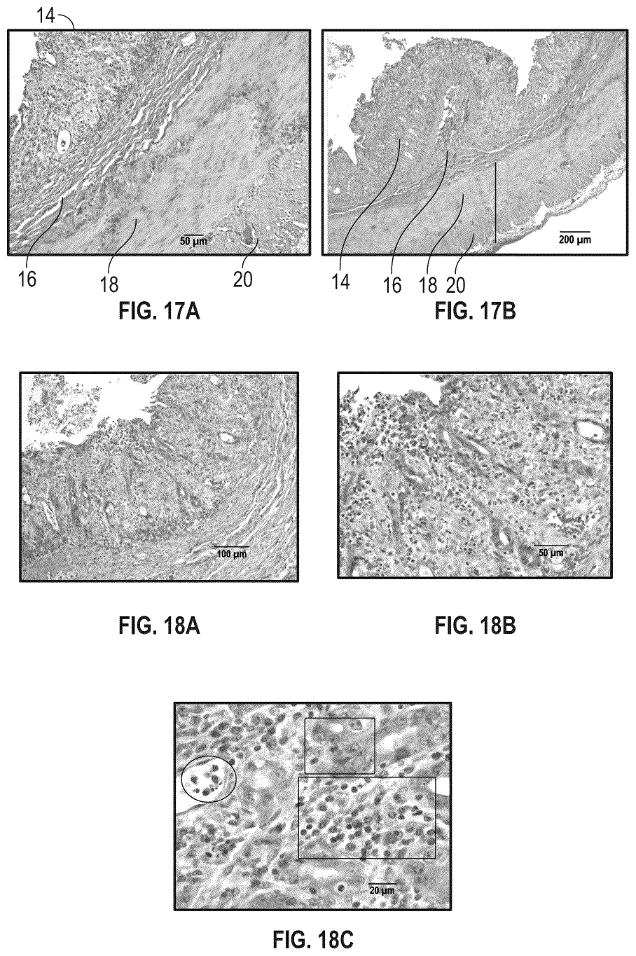

FIGS. 17A and 17B illustrate the appearance of the mucosa 24 hours after the treatment with five electrolytic electroporation pulses of 500 V and 100 μF. FIGS. 17A and 17B illustrate different locations in the tissue. Five pulses appear to cause more substantial ablation of the mucosa; however, the muscularis is intact, and the ablation is confined to the mucosa.

FIGS. 18A-18C illustrate the histology of the tissue treated with five electrolytic electroporation pulses 24 hours after the treatment. The sections have regions of mucosa necrosis characterized as subacute injury of the mucosal epithelium with concurrent regeneration (already after 24 hours) as evidenced by the proliferation of the basophilic immature crypt cells. There is persistent crypt dilatation with or without necrotic debris. There is minimal to mild infiltration of mixed inflammatory cells including macrophages, lymphocytes, and a few neutrophils. The small circle of FIG. 18C illustrates cellular debris in the crypts. The small square of FIG. 18C illustrates regenerative crypt epithelial cells. The large square of FIG. 18C illustrates mixed inflammatory cells infiltrate consisting of macrophages (large cells with big nuclei); small dark cells are lymphocytes and bright cytoplasmic cells with dumb-bell shaped nuclei are PMNs (polymorphonuclear leukocytes, neutrophils, and/or eosinophils).

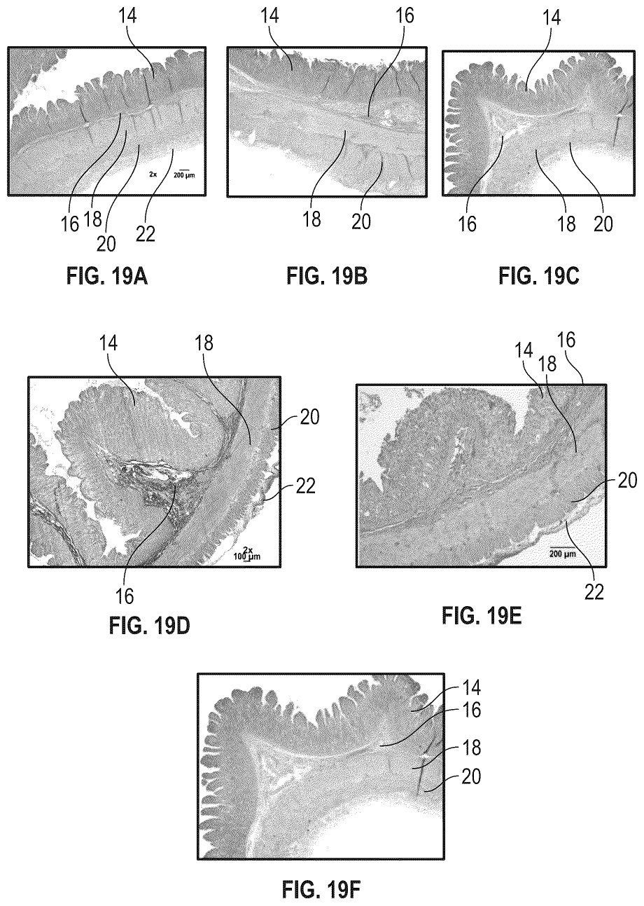

FIG. 19A illustrates electrolytic electroporation ablated tissue 72 hours (three days) after the treatment with one pulse. FIG. 19B illustrates electrolytic electroporation ablated tissue 72 hours (three days) after the treatment with three pulses. FIG. 19C illustrates electrolytic electroporation ablated tissue 72 hours (three days) after the treatment with five pulses. It is evident that regardless of the number of pulses the tissue is completely regenerated after three days. FIG. 19D illustrates mucosa of a control sample, FIG. 19E shows tissue treated with five electrolytic electroporation pulses after 24 hours, and FIG. 19F illustrates tissue treated with five electrolytic electroporation pulses after 72 hours. The comparison demonstrates that five electrolytic electroporation pulses ablate the mucosa without affecting the muscularis and this ablation is evident after 24 hours. However, 72 hours after treatment with the five electrolytic electroporation pulses and the ablation after 24 hours, the tissue is completely regenerated, as expected when the ablation has not damaged the extracellular scaffold. This experiment demonstrates that the design of an electrolytic electroporation treatment protocol for ablation of tissue so that it can regenerate.

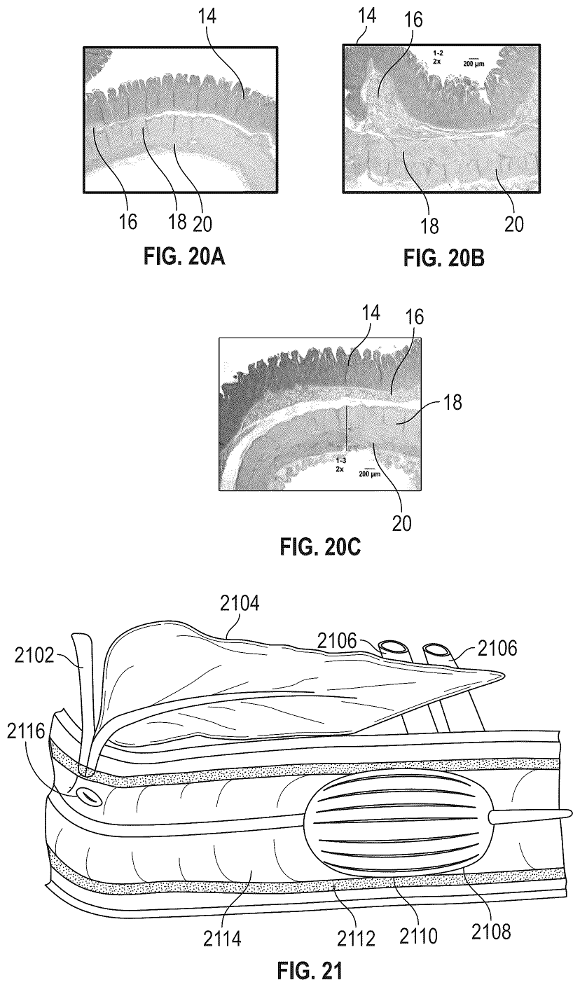

FIGS. 20A-20C illustrate electrolytic electroporation ablated tissue eight days after the electrolytic electroporation treatment. FIG. 20A illustrates the appearance after one pulse. FIG. 20B illustrates the appearance after two pulses. FIG. 20C illustrates the appearance after five pulses. It is evident that regardless of the number of pulses the tissue is completely regenerated at eight days as expected when the ablation has not damaged the extracellular scaffold. Furthermore, ablation depth may be controlled to cause tissue regeneration without scar or fibrosis, absence of collagen bands, or ulceration out to 30 days. This experiment demonstrates the design of an electrolytic electroporation treatment protocol for ablation of tissue so that it has the ability to regenerate, e.g., using electrolytic electroporation for regeneration of tissue. From this experiment, a process of electrolytic electroporation and a recovery of tissue may be confirmed.

### Mathematical Model II

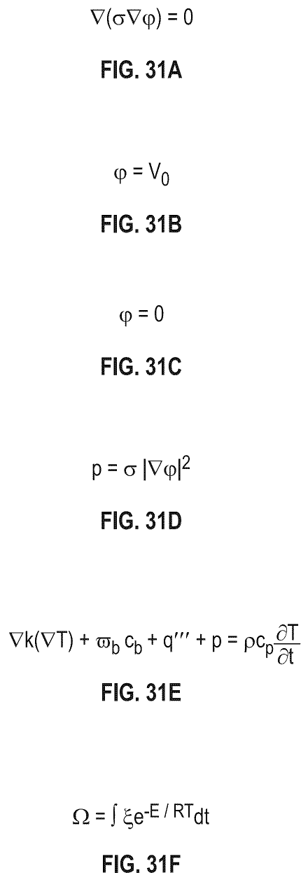

A mathematical model was developed to employ an analysis of the electric field distribution, the thermal energy dissipated, and the temperature distribution. This mathematical model may be used to determine electric fields for use in examples of the application of electrolysis and electroporation described herein. An electrical potential associated with an electroporation pulse is determined by solving the Laplace equation of FIG. 31A for the potential distribution, where φ is the electrical potential and σ is the electrical conductivity. The electrical boundary condition of the tissue that is in contact with the leftmost electrode(s) on which the electroporation voltage, V0, is represented as an equation of FIG. 31B. The electrical boundary condition at the interface of the second electrode(s) is represented as an equation of FIG. 31C. The boundaries where the analyzed domain is not in contact with an electrode are treated as electrically insulative. The solution of these equations, with the relevant geometry and boundary conditions, gives the electric field distribution in the tissue. By solving for the electric field, the solution for the associated Joule heating (p), which is the heat generation rate per unit volume caused by the electrical field dissipation represented as an equation of FIG. 31D may be computed. This term is added to the original Pennes equation to represent the heat generated from the electroporation procedure as shown in FIG. 31E, where k is the thermal conductivity of the tissue, T is the temperature, w_{b} is the blood perfusion, c_{b} is the heat capacity of the blood, Tₐ is the arterial temperature, q′″ is the metabolic heat generation, ρ is the tissue density, and cₚ is the heat capacity of the tissue. The goal of this part of the analysis is to determine the temperature distribution in the tissue. Thermal damage is a temperature and time dependent process, where thermal damage is described by an Arrhenius type equation of FIG. 31F. In some examples, thermal damage may begin at temperatures higher than 42° C. However, up to the temperature range 50° C. and 60° C., the damage may be relatively low and it occurs for times of exposure between minutes and hours.

### Example 2—Animal Model

In this animal model, a female, 100 kg Yorkshire pig was used. The animal study established qualitative correlation between a number of pulses and a depth of ablation. A medical procedure similar to the medical procedure illustrated in Example 1—Animal Model was used, thus a detailed description of the medical procedures that have been previously described, such as referring to FIGS. 7A and 7B, is therefore not repeated herein for brevity.

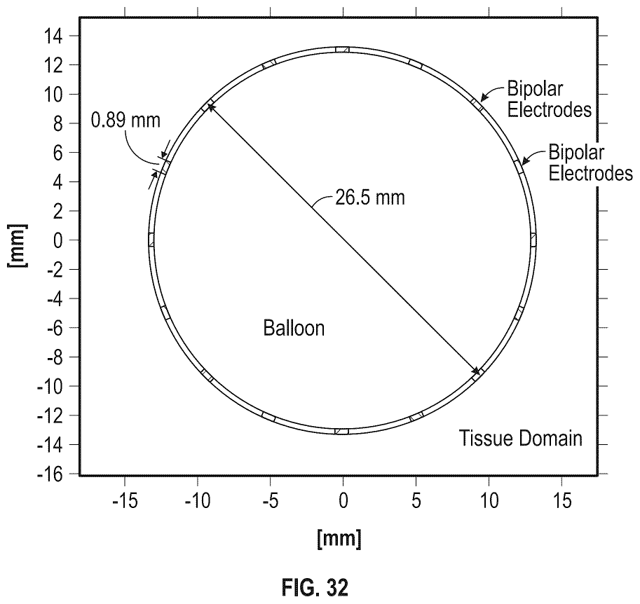

In this example, an electrolytic electroporation electrode array, including 16 electrodes of eight cathodes and eight anodes, was then deployed using a catheter (e.g., the catheter 200) to achieve adequate tissue apposition. The geometry analyzed is shown in FIG. 32. A width of each electrode is 0.89 mm, and a gap between the electrodes 4.31 mm. The diameter of the balloon is 26.5 mm. The catheter was guided to the manually advanced into the mid portion of the jejunum, and electrolytic electroporation DMRe was then performed from the mid jejunum back toward the proximal duodenum, working distal to proximal. A 500V exponential decay waveform that was modeled to achieve a 1000 kv/cm² electrolytic electroporation field of ablation may be applied to the tissue via the axial electrode array. Four treatment sites were performed in the duodenum and four treatment sites were performed in the jejunum measuring approximately 3 cm in length at each treatment site. In total, eight total ablations were performed: two with one pulse, four with five pulses, and two with 10 pulses. The ablation sites were marked with endovascular clips on the mesenteric surface of the small bowel at the mid-point of each ablation zone for site identification. Overlapping zones of ablation were simulated to understand potential safety issues and treatment effect with the application of 10 pulses.

Euthanasia was then performed at five to six hours after the electrolytic electroporation ablation for each animal, and the proximal small bowel was resected and explanted. The treatment zones were then identified and the tissue was processed by obtaining both axial and longitudinal sections that were then fixed in formalin for 24 hours, processed, and fixed in paraffin blocks. Tissue samples were then stained with hematoxylin and eosin and Masson's trichrome stain. Sections were then evaluated to identify the depth and extent of ablation. Further, annotation was provided by an expert gastrointestinal pathologist to determine the overall circumferential completeness of the ablation to correlate to the array to obtain a ratio of circumferential ablation (e.g., an amount of circumference of tissue in the target region that has been affected). There were no changes in the hemodynamics of the animal during the ablations. Mild muscle contractions were observed that did not warrant the administration of intra-procedural muscle paralytics.

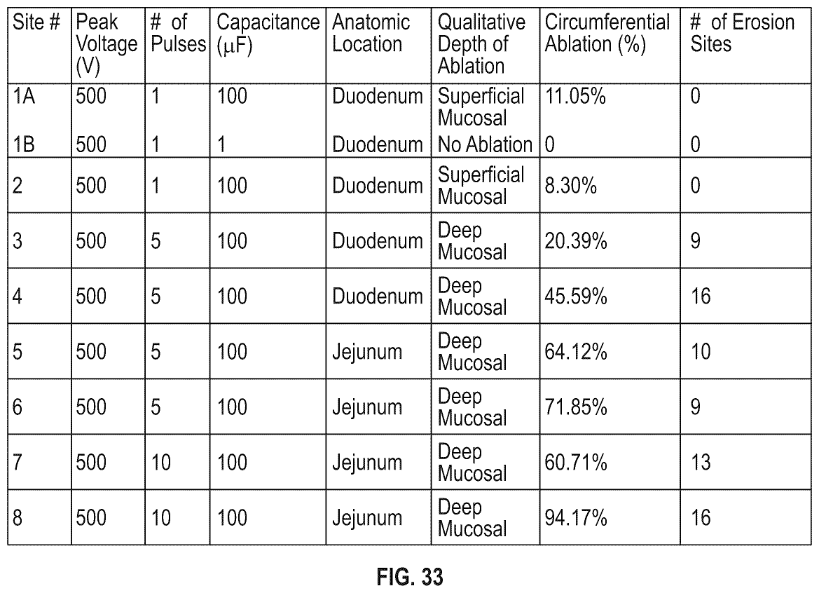

FIG. 33 is a table presenting an outcome of an experiment. The voltage was kept at 500 V. The capacitance was varied between 100 μF and 1 μF at Sites 1A and 1B. For the other sites, the capacitance was kept at 100 μF. A voltage of 500 V (peak) was used. The number of pulses varied from one to five to 10. When the capacitance was 1 μF, there was no ablation. Thus, the capacitance difference affects whether the ablation may be caused. When the capacitance was constant at 100 μF, the number of pulses is likely to contribute to depth of ablation. For example, when the number of pulses is one in this example, the depth of ablation at many sites is limited to superficial mucosal. On the other hand, when the number of pulses is five or 10 in this example, the depth of ablation reaches deep mucosal ablation. Furthermore, when the number of pulses is five in this example, 50% circumferential endoluminal ablation is achieved, whereas 75% circumferential endoluminal ablation is achieved when the number of pulses is 10 in this example. The results of the electrolytic electroporation show that as the number of pulses applied to target tissue increases, a depth of ablation and an amount of ablation within a particular circumference increase. Additionally, the circumference of tissue affected by ablation increases with increasing dosimetry (e.g., number of pulses).

### Example 3—Animal Model

In this animal model, female, ˜60 kg Yorkshire pigs were used. The animal study further established quantitative correlation between a number of pulses and a depth of ablation. A medical procedure similar to the medical procedure illustrated in Examples 1 and 2-Animal Model was used, thus a detailed description of the medical procedures that have been previously described, such as referring to FIGS. 7A and 7B, is therefore not repeated herein for brevity.

In this example, an electrolytic electroporation electrode array, including 16 titanium electrodes affixed to its outer surface was then deployed using a catheter (e.g., the catheter 200) to achieve adequate tissue apposition. The catheter was guided to the manually advanced into the mid portion of the jejunum, and electrolytic electroporation DMRe was then performed from the mid jejunum back toward the proximal duodenum, working distal to proximal. A total of six treatment sites were performed per animal: two in the duodenum and four in the jejunum. Each treatment site was marked with an endovascular clip on the mesenteric surface of the small bowel at the mid-point of each treatment site. The total length of each treatment site measured approximately 3 cm. Euthanasia was then performed at 24 hours, three days, eight days, and 30 days after the electrolytic electroporation ablation for each animal, and the proximal small bowel was resected and explanted. The treatment zones were then identified and the tissue was processed by obtaining both axial and longitudinal sections that were then fixed in formalin overnight, processed, and fixed in paraffin blocks. Tissue samples were then stained with hematoxylin and eosin and Masson's trichrome stain. Sections were then evaluated to identify the depth and extent of ablation. Further, annotation was provided by an expert gastrointestinal pathologist to determine the overall circumferential completeness of the ablation to correlate to the array to obtain a ratio of circumferential ablation.

### Tissue Regeneration Study at 24 Hours, Three Days, Eight Days, and 30 Days

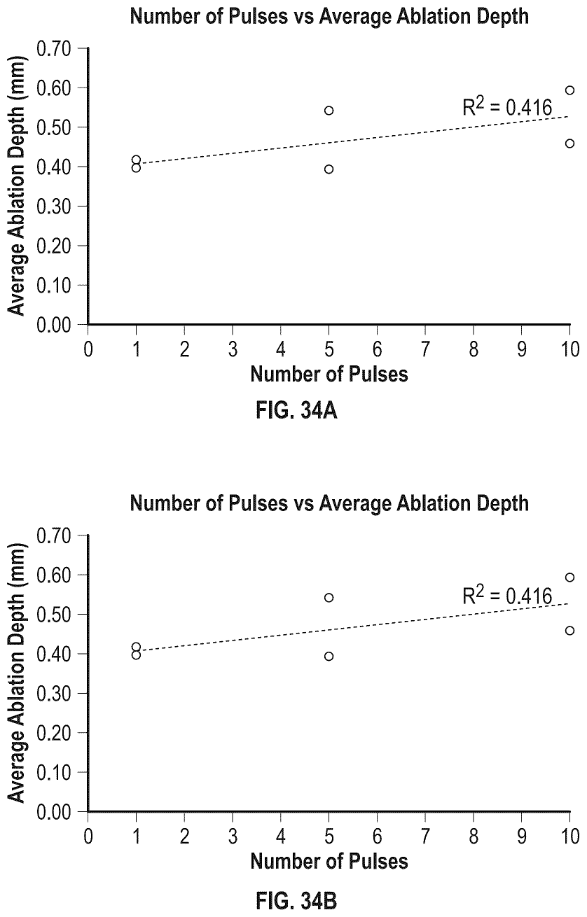

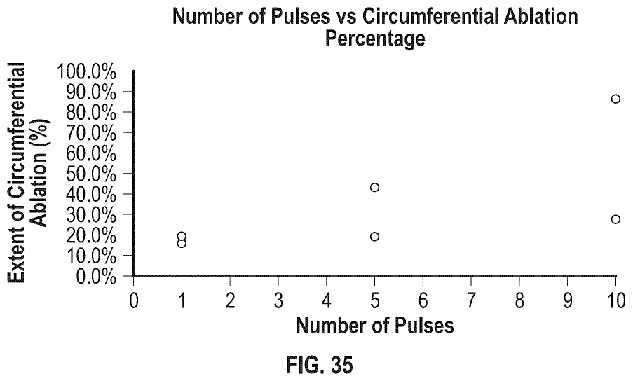

FIGS. 34A and 34B are schematic illustrations showing relationships between numbers of pulses and average ablation depths of jejunum and duodenum, respectively, in examples of systems described herein. FIG. 35 is a schematic illustration showing relationships between numbers of pulses and circumferential ablation percentage in examples of systems described herein. The samples collected 24 hours post-procedure showed circumferential ablation at 19-40% indicating a partial healing of the mucosa at this time point. Similarly, longitudinal percent ablation along the length of the mucosa demonstrated 13-34% ablation. There was an efficacy-dependent correlation between number of pulses (i.e., dose intensity) and percentage circumferential ablation achieved as shown in FIGS. 34A, 34B, and 35. Ablation depth ranged from 0.4 mm to 0.6 mm indicating limited depth to the submucosa. There was a positive correlation between number of pulses and depth. Maximum ablation depth ranged from 0.6 mm to 1.2 mm with a peak around 1.0 mm at 10 pulses. The results of the electrolytic electroporation show that as the increase in a number of pulses, a depth of ablation, and a ratio of ablation increase.

Samples collected at three and eight days post-procedure showed no evidence of tissue injuries and histologically appear the same as the control sample. Villi architecture appears normal and full recovery of mucosa was observed even at the highest number of pulses. External mild serosal adhesions were seen with some samples suggestive of physical handling of the bowel during gastrostomy. There were no transmural injuries observed nor any collagen deposits that can be an early sign of fibrosis. Similarly, there was no evidence of epithelial injury and complete healing has occurred at 30 days. Gross visualization of treatment sites showed no signs of perforation or strictures. Villous architecture appears normal and there is an absence of inflammation at the site of treatment. Some samples showed an abundance of microvascular proliferation near the mucosa at 30 days indicating healthy regeneration of the duodenal mucosa. This was exclusively observed in sites treated with 10 pulses. The results of the electrolytic electroporation show that as the increase in a number of pulses, a depth of ablation, and a ratio of ablation increase that result in healthy regeneration of the duodenal mucosa.

This ability can be extended to implantation and/or transplantation of stem cells, molecules, genetic material, organoids, or other desired cells in the electrolytic electroporation ablated tissue prior and during the period from ablation to regeneration.

Examples provided herein of both the design of delivery systems and the clinical applications are not the limit of the uses of the combination of electroporation and electrolysis while retaining extracellular matrices. Many configurations of delivery systems exist, as well as applications that would benefit from the use of the technology described herein.

It is to be appreciated that any one of the above embodiments or processes may be combined with one or more other embodiments and/or processes or be separated and/or performed among separate devices or device portions in accordance with the present systems, devices and methods.

Finally, the above discussion is intended to be merely illustrative of the present devices, apparatuses, systems, and methods and should not be construed as limiting the appended claims to any particular embodiment or group of embodiments. Thus, while the present disclosure has been described in particular detail with reference to exemplary embodiments, it should also be appreciated that numerous modifications and alternative embodiments may be practiced without departing from the broader and intended spirit and scope of the present disclosure as set forth in the claims that follow. Accordingly, the specification and drawings are to be regarded in an illustrative manner and are not intended to limit the scope of the appended claims.

1. A method comprising:
positioning at least one electrode proximate tissue of a patient;
applying an electric field to at least a portion of the patient using the at least one electrode, the electric field configured to permeabilize cell membranes in a targeted tissue of the patient, thereby generating permeabilized cells, wherein the permeabilized cells are within an area targeted for ablation; and
performing electrolysis to generate products of electrolysis in the targeted tissue to ablate the permeabilized cells while leaving intact an extracellular matrix in the targeted tissue.

2. The method of claim 1, wherein said positioning comprises delivering a catheter to the patient, the catheter including the at least one electrode.

3. The method of claim 2, wherein said applying the electric field comprises applying a voltage between the at least one electrode and a return electrode placed proximate the patient.

4. The method of claim 2, wherein the catheter includes a pair of electrodes including the at least one electrode, and wherein said applying the electric field comprises applying a voltage between the pair of electrodes.

5. The method of claim 1, wherein the extracellular matrix provides a scaffold for tissue regeneration.

6. The method of claim 1, wherein said at least one electrode proximate tissue of the patient is disposed to retain stem cells in a crypt of mucosa of the tissue.

7. (canceled)

8. (canceled)

9. The method of claim 1, wherein a number of pulses is selected based on a depth of ablation.

10. (canceled)

11. The method of claim 1, wherein the targeted tissue is a mucosal layer of a small intestine.

12. The method of claim 11, wherein the electric field at an interface between a mucosal and submucosal layer has a field strength less than that used for irreversible electroporation.

13. (canceled)

14. (canceled)

15. The method of claim 1, further comprising introducing additional cells to the targeted tissue, through transplantation, migration, or combinations thereof, wherein the additional cells facilitate regeneration.

16. A system for performing electrolytic electroporation regeneration comprising:
a delivery system with having a plurality of electrodes configured to contact an area of tissue targeted for ablation;
a controller configured to control a charge applied to the electrodes, the controller configured to:
induce a voltage to generate permeabilized cells in the area of tissue targeted for ablation; and
induce a current to generate products of electrolysis to cause ablation of the permeabilized cells while leaving an extracellular matrix of at least some of the permeabilized cells intact to allow for regeneration of the tissue including the permeabilized cells.

17. The system of claim 16, wherein the delivery system comprises a catheter and wherein a distal portion of the catheter comprises an expandable member configured to contact the area of tissue targeted for ablation, the expandable member including the plurality of electrodes.

18. The system of claim 17, wherein the plurality of electrodes extend from a proximal portion to a distal portion of an extendable member.

19. The system of claim 16, wherein the plurality of electrodes is between 12 electrodes and 16 electrodes.

20. The system of claim 16, wherein the plurality of electrodes are disposed in a manner to retain stem cells in a crypt of mucosa of the tissue.

21. (canceled)

22. (canceled)

23. The system of claim 16, wherein the controller is configured to select a number of pulses based on a depth of ablation.

24. A non-transitory computer-readable storage media, the computer-readable storage media including instructions that when executed by a controller, cause the controller to:
induce a voltage to generate permeabilized cells in an area of tissue targeted for ablation; and
induce a current to generate products of electrolysis to cause ablation of the permeabilized cells while leaving an extracellular matrix of at least some of the permeabilized cells intact to allow for regeneration of the tissue including the permeabilized cells.

25. (canceled)

26. (canceled)

27. The non-transitory computer-readable storage media of claim 24, wherein the instructions further cause the controller to select the number of pulses based on a depth of ablation.

28. The method of claim 1, wherein said performing electrolysis to generate products of electrolysis in the targeted tissue comprises setting at least one of a time duration or a quantity of electrolysis products to leave the extracellular matrix of at least some of the permeabilized cells intact.

29. The system of claim 16, wherein the controller is configured to induce the current to generate products of electrolysis by setting at least one of a time duration or a quantity of electrolysis products to leave the extracellular matrix of at least some of the permeabilized cells intact.

30. The non-transitory computer-readable storage media of claim 24, wherein the controller is configured to induce the current to generate products of electrolysis by setting at least one of a time duration or a quantity of electrolysis products to leave the extracellular matrix of at least some of the permeabilized cells intact.
